(19) United States
(12) Patent Application Publication
(10) Pub. No.: US 2024/0254181 A1
(43) Pub. Date: Aug. 1, 2024

(54) RECOMBINANT ADENO-ASSOCIATED VIRUS PRODUCTS AND METHODS FOR TREATING DYSTROGLYCANOPATHIES AND LAMININ-DEFICIENT MUSCULAR DYSTROPHIES

(71) Applicant: RESEARCH INSTITUTE AT NATIONWIDE CHILDREN'S HOSPITAL, Columbus, OH (US)

(72) Inventor: Paul Taylor Martin, Bexley, OH (US)

(21) Appl. No.: 17/254,055

(22) PCT Filed: Jun. 18, 2019
(86) PCT No.: PCT/US19/37769

§ 371 Date: Dec. 18, 2020

Related U.S. Application Data
(60) Provisional application No. 62/686,522, filed on Jun. 18, 2018.

(51) Int. Cl. C07K 14/485; A61K 48/00; A61P 21/00; C07K 14/47; C07K 14/78; C12N 15/86

(52) U.S. Cl. CPC C07K 14/485; A61K 48/0058; A61P 21/00; C07K 14/4707; C07K 14/78; C12N 15/86; C07K 2319/00; C12N 2750/14143; C12N 2830/008

(57) ABSTRACT

Products and methods for treating dystroglycanopathies and laminin-deficient muscular dystrophies are provided. In the methods, a protein including a linker domain, such as the heparin-binding domain of Heparin-Binding Epidermal Growth Factor-Like Growth Factor (HBEGF), is delivered to patients.

This application is a national phase application of International Application No. PCT/US19/37769, filed on Jun. 18, 2019, which claims priority benefit to U.S. Provisional Patent Application No. 62/686,522, filed Jun. 18, 2018, which are incorporated by reference herein in its entirety.

This invention was made with government support under AR070604 awarded by The National Institutes of Health. The government has certain rights in the invention.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 53147A_SubSeqlisting.txt; Size: 126,129 bytes, created; Mar. 15, 2021.

### FIELD OF THE INVENTION

Products and methods for treating dystroglycanopathies and laminin-deficient muscular dystrophies are provided. In the methods, a protein including a linker domain, such as the heparin-binding domain of Heparin-Binding Epidermal Growth Factor-Like Growth Factor (HBEGF), is delivered to patients. This linker protein assists in targeting a transgene to the extracellular matrix (ECM) of a muscle cell.

### BACKGROUND

Muscular dystrophies (MDs) are a group of genetic diseases. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

Congenital muscular dystrophy (CMD) describes a group of MDs in which the loss of muscle structural components results in neonatal hypotonia and progressive skeletal muscle weakness. These disorders are often associated with significant extramuscular complications, including brain and eye developmental defects, cognitive impairment, seizures, and respiratory and cardiac abnormalities, requiring regular medical management by a multidisciplinary team. The estimated incidence of CMD is 1 in 21,500 live births worldwide. Despite the gravity of these disorders, there are currently no approved and effective therapies. Dystroglycanopathies and merosin-deficient CMD Type 1A (MDC1A) are two of the most common forms of CMD [Sframeli et al., Neuromuscul Disord., 27(9): 793-803 (2017)].

Dystroglycanopathies are caused by mutations in any of eighteen or more genes required for glycosylating α-dystroglycan. Proper glycosylation allows α-dystroglycan to bind components of the extracellular matrix (ECM). α-Dystroglycan, in turn, anchors to the sarcolemma by binding β-dystroglycan, a transmembrane protein. The number of susceptible genes makes impossible the development of a single gene-replacement therapy for dystroglycanopathy. Examples of dystroglycanopathies include the following. Walker-Warburg Syndrome (WWS) involves genetic mutations in B3GLNT2, B4GAT1, DAG1, FKRP, FKTN, GMPPB, ISPD, or LARGE. Muscle Eye Brain disease (MEB) involves genetic mutations in B3GLNT2, B4GAT1, DAG1, FKRP, FKTN, GMPPB, ISPD, or LARGE. Fukuyama CMD involves mutations in the FKTN gene. A group of congenital muscular dystrophies with cognitive impairment results from mutations in FKRP, LARGE, POMT1, POMT2, or POMGNT1. A group of CMDs without cognitive impairment are a result of genetic mutations in FKRP or FKTN. Limb Girdle Muscular Dystrophies LGMD 2I, 2K, 2M, 2N and 2O are associated with glycosylation abnormalities involving genetic mutations in FKRP, FKTN, POMGNT1, POMT1, or POMT2. Limb Girdle Muscular Dystrophies LGMD 2T and 2U are respectively a result of genetic mutations in GMPPB and ISPD. Other mutated genes in dystroglycanopathies include DOLK, DPM1, DPM2, DPM3, GTDC2/AG061, TMEM5, and SK196.

MDC1A is caused by mutations in the LAMA2 gene, encoding the key ECM protein, laminin-α2, which binds glycosylated α-dystroglycan at the sarcolemma. The full LAMA2 gene is over 9,000 base pairs in length.

A study by Reinhard and colleagues [Reinhard et al., Sci Transl Med., 9(396), (2017)] involved germline expression of fused domains from laminin-α4 and mini-agrin to incompletely ameliorate disease symptoms in the dyW/dyW mouse model of MDC1A.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

There remains a need in the art for treatments for CMDs such as dystroglycanopathies and MDC1A.

### SUMMARY

Provided herein are methods and products for treatment of CMDs such as dystroglycanopathies and laminin-deficient muscular dystrophies. The products include therapeutic proteins and rAAV encoding a disclosed therapeutic protein.

A polynucleotide is provided encoding a protein comprising:
a) a first domain comprising the heparin-binding domain of Heparin-Binding Epidermal Growth Factor-Like Growth Factor (HBEGF), and a second domain comprising the G1-G5 domain of the human laminin alpha 2 (LAMA2) gene;
b) a first domain comprising the heparin-binding domain of HBEGF and the EGF-like domain of HBEGF, and a second domain comprising the G1-G5 domain of the human LAMA2 gene;
c) a first domain comprising the heparin-binding domain of HBEGF, and a second domain comprising the G3-G5 domain of the human LAMA2 gene;
d) a first domain comprising the heparin-binding domain of HBEGF and the EGF-like domain of HBEGF, and a second domain comprising the G3-G5 domain of the human LAMA2 gene,
e) a first domain comprising the heparin-binding domain of HBEGF and a second domain comprising DAG1alpha or
f) a first domain comprising the heparin-binding domain of HBEGF and the EGF-like domain of HBEGF, and a second domain comprising DAG1 alpha.

In one embodiment, the provided polynucleotides encode a protein, wherein the first domain of the protein is encoded by the nucleotide sequence of SEQ ID NO: 13 or SEQ ID NO: 14, and the second the second domain of the protein is encoded by the nucleotide sequence of SEQ ID NO: 15, SEQ ID NO: 16 or SEQ ID NO: 17.

For example, the provided polynucleotides comprises the nucleotide sequence of SEQ ID NO: 13 and SEQ ID NO: 15, or comprise the nucleotide sequence of SEQ ID NO: 13 and SEQ ID NO: 16, or comprise the nucleotide sequence of SEQ ID NO: 14 and SEQ ID NO: 15, or comprise the nucleotide sequence of SEQ ID NO: 14 and SEQ ID NO: 16, or comprise the nucleotide sequence of SEQ ID NO: 13 and SEQ ID NO: 17 or comprise the nucleotide sequence of SEQ ID NO: 14 and SEQ ID NO: 17.

In one embodiment, the provided polynucleotides comprise one of the following: i) the nucleotide sequence set forth in FIG. 3, ii) a nucleotide sequence comprising nucleotides 14 to 3235 set out in FIG. 3, iii) the nucleotide sequence of SEQ ID NO: 1, or iv) a nucleotide sequence encoding the amino acid sequence of SEQ ID NO: 19.

In another embodiment, the provided polynucleotides comprise one of the following: i) the nucleotide sequence set forth in FIG. 4, ii) a nucleotide sequence comprising nucleotides 14 to 3361 set forth in FIG. 4, iii) the nucleotide sequence of SEQ ID NO: 3 or iv) a nucleotide sequence encoding the amino acid sequence of SEQ ID NO: 20.

In a further embodiment, the provided polynucleotides comprise one of the following: i) the nucleotide sequence set forth in FIG. 5, ii) a nucleotide sequence comprising nucleotides 14 to 1930 set forth in FIG. 5, iii) the nucleotide sequence of SEQ ID NO: 5 or iv) a nucleotide sequence encoding the amino acid sequence of SEQ ID NO: 21.

In another embodiment, the provided polynucleotides comprise one of the following: i) the nucleotide sequence set forth in FIG. 6, ii) a nucleotide sequence comprising nucleotides 14 to 2056 set forth in FIG. 6, iii) the nucleotide sequence of SEQ ID NO: 7 or iv) a nucleotide sequence encoding the amino acid sequence of SEQ ID NO: 22.

In an embodiment, the provided polynucleotides comprise one of the following: i) the nucleotide sequence set forth in FIG. 7, ii) a nucleotide sequence comprising nucleotides 14 to 1360 set forth in FIG. 7, iii) the nucleotide sequence of SEQ ID NO: 9 or iv) a nucleotide sequence encoding the amino acid sequence of SEQ ID NO: 23.

In another embodiment, the provided polynucleotides comprise one of the following: i) the nucleotide sequence set forth in FIG. 8, ii) a nucleotide sequence comprising nucleotides 14-1486 set forth in FIG. 8, iii) the nucleotide sequence of SEQ ID NO: 11 or iv) a nucleotide sequence encoding the amino acid sequence of SEQ ID NO: 24.

Therapeutic proteins encoded by any of the provided polynucleotides are also provided. For example, the provided proteins comprise the amino acid sequence of SEQ ID NO: 19, SEQ ID NO: 20, SEQ ID NO: 21, SEQ ID NO: 22, SEQ ID NO: 23 or SEQ ID NO: 24.

In addition, the disclosure provides recombinant host cells comprising any of the polynucleotide described herein. In exemplary embodiments, the host cells, the polynucleotides are operatively linked to a transcriptional control element and these host cells express any of the polynucleotides disclosed herein. For example, the host cells are Chinese hamster ovary (CHO) cell or human HEK293 cell.

Further provided are recombinant adeno-associate virus (rAAV), wherein the genome of the rAAV comprises any of the polynucleotide described herein. For example, provided are rAAV, wherein the genome of the rAAV comprises a polynucleotide sequence of SEQ ID NO: 1, SEQ ID NO: 3, SEQ ID NO: 5, SEQ ID NO: 7, SEQ ID NO: 9 or SEQ ID NO: 11. In exemplary embodiments, the rAAV genome further comprises a muscle-specific transcriptional control element, such as a CMV promoter (SEQ ID NO: 18), MCK, NHCK, LAMA2 or tMCK. Any of the rAAV described herein comprise the AAV9, AAV10, AAVrh74, AAV8 or AAV6 capsid.

Also provided are rAAV, wherein the genome of the rAAV comprises nucleotides 3590 to 8215 of SEQ ID NO: 2, nucleotides 3590 to 8341 of SEQ ID NO: 4, nucleotides 3609 to 6929 of SEQ ID NO: 6, nucleotides 3590 to7036 of SEQ ID NO: 8, nucleotides 3590 to 6340 of SEQ ID NO: 10, nucleotides 3590 to 6049 of SEQ ID NO: 12, the nucleotide sequence set out in FIG. 13, or the nucleotide sequence set out in FIG. 14.

rAAV particles comprising any of the rAAV described herein are also provided. The disclosure also provides for compositions comprising any of the polynucleotides disclosed herein, any of the rAAV disclosed herein, any of the rAAV particles disclosed herein or any of the proteins disclosed herein.

Provided are methods for treating a laminin-deficient muscular dystrophy comprising administering to a patient in need thereof a rAAV, wherein the genome of the rAAV comprises a polynucleotide encoding a protein comprising:
a) a first domain comprising the heparin-binding domain of HBEGF, and a second domain comprising the G1-G5 domain of the human LAMA2 gene;
b) a first domain comprising the heparin-binding domain of HBEGF and the EGF-like domain of HBEGF, and a second domain comprising the G1-G5 domain of the human LAMA2 gene;
c) a first domain comprising the heparin-binding domain of HBEGF, and a second domain comprising the G3-G5 domain of the human LAMA2 gene; or
d) a first domain comprising the heparin-binding domain of HBEGF and the EGF-like domain of HBEGF, and a second domain comprising the G3-G5 domain of the human LAMA2 gene.

For example, the methods of treating a laminin-deficient muscular dystrophy comprise administering to a patient in need thereof any of the following: any of the polynucleotides disclosed herein which encode a protein comprising LAMA2(G1-G5) or LAMA2(G3-G5) as the second domain, any of the rAAV or rAAV particles disclosed herein which comprise a polynucleotide encoding a protein comprising LAMA2(G1-G5) or LAMA2(G3-G5) as the second domain.

Further provided are methods for treating a laminin-deficient muscular dystrophy comprising administering to a patient in need thereof a protein comprising:
a) a first domain comprising the heparin-binding domain of HBEGF, and a second domain comprising the G1-G5 domain of the human LAMA2 gene;
b) a first domain comprising the heparin-binding domain of HBEGF and the EGF-like domain of HBEGF, and a second domain comprising the G1-G5 domain of the human LAMA2 gene;
c) a first domain comprising the heparin-binding domain of HBEGF, and a second domain comprising the G3-G5 domain of the human LAMA2 gene; or
d) a first domain comprising the heparin-binding domain of HBEGF and the EGF-like domain of HBEGF, and a second domain comprising the G3-G5 domain of the human LAMA2 gene.

For example, the methods for treating a treating laminin-deficient muscular dystrophy comprise administering a protein to a patient in need thereof, wherein the protein comprises the amino acid sequence of SEQ ID NO: 19, SEQ ID NO: 20, SEQ ID NO: 21 or SEQ ID NO: 22.

Also provided are compositions for treating a laminin-deficient muscular dystrophy comprising a rAAV, wherein the genome of the rAAV comprises a polynucleotide encoding a protein comprising:
a) a first domain comprising the heparin-binding domain of HBEGF, and a second domain comprising the G1-G5 domain of the human LAMA2 gene;
b) a first domain comprising the heparin-binding domain of HBEGF and the EGF-like domain of HBEGF, and a second domain comprising the G1-G5 domain of the human LAMA2 gene;
c) a first domain comprising the heparin-binding domain of HBEGF, and a second domain comprising the G3-G5 domain of the human LAMA2 gene; or
d) a first domain comprising the heparin-binding domain of HBEGF and the EGF-like domain of HBEGF, and a second domain comprising the G3-G5 domain of the human LAMA2 gene.

For example, the compositions for treating a laminin-deficient muscular dystrophy comprise any of the following: any of the polynucleotides disclosed herein which encode a protein comprising LAMA2(G1-G5) or LAMA2(G3-G5) as the second domain, any of the rAAV or rAAV particles disclosed herein which comprise a polynucleotide encoding a protein comprising LAMA2(G1-G5) or LAMA2(G3-G5) as the second domain.

Further provided are compositions for treating laminin-deficient muscular dystrophies comprising a protein comprising:
a) a first domain comprising the heparin-binding domain of HBEGF, and a second domain comprising the G1-G5 domain of the human LAMA2 gene;
b) a first domain comprising the heparin-binding domain of HBEGF and the EGF-like domain of HBEGF, and a second domain comprising the G1-G5 domain of the human LAMA2 gene;
c) a first domain comprising the heparin-binding domain of HBEGF, and a second domain comprising the G3-G5 domain of the human LAMA2 gene; or
d) a first domain comprising the heparin-binding domain of HBEGF and the EGF-like domain of HBEGF, and a second domain comprising the G3-G5 domain of the human LAMA2 gene.

For example, the compositions for treating a treating laminin-deficient muscular dystrophy comprise a protein, wherein the protein comprises the amino acid sequence of SEQ ID NO: 19, SEQ ID NO: 20, SEQ ID NO: 21 or SEQ ID NO: 22.

The disclosure also provides for a use of a rAAV for the preparation of a medicament for treating a laminin-deficient muscular dystrophy in a patient in need thereof, wherein the genome of the rAAV comprises a polynucleotide encoding a protein comprising:
a) a first domain comprising the heparin-binding domain of HBEGF, and a second domain comprising the G1-G5 domain of the human LAMA2 gene;
b) a first domain comprising the heparin-binding domain of HBEGF and the EGF-like domain of HBEGF, and a second domain comprising the G1-G5 domain of the human LAMA2 gene;
c) a first domain comprising the heparin-binding domain of HBEGF, and a second domain comprising the G3-G5 domain of the human LAMA2 gene; or
d) a first domain comprising the heparin-binding domain of HBEGF and the EGF-like domain of HBEGF, and a second domain comprising the G3-G5 domain of the human LAMA2 gene.

For example, the disclosure also provides for use of any of the following: any of the polynucleotides disclosed herein which encode a protein comprising LAMA2(G1-G5) or LAMA2(G3-G5) as the second domain, any of the rAAV or rAAV particles disclosed herein which comprise a polynucleotide encoding a protein comprising LAMA2(G1-G5) or LAMA2(G3-G5) as the second domain for the preparation of a medicament for treating a laminin-deficient muscular dystrophy in a patient in need thereof.

Further provided are use of a protein for the preparation of a medicament for treating a laminin-deficient muscular dystrophy in a patient in need thereof, wherein the protein comprises:
a) a first domain comprising the heparin-binding domain of HBEGF, and a second domain comprising the G1-G5 domain of the human LAMA2 gene;
b) a first domain comprising the heparin-binding domain of HBEGF and the EGF-like domain of HBEGF, and a second domain comprising the G1-G5 domain of the human LAMA2 gene;
c) a first domain comprising the heparin-binding domain of HBEGF, and a second domain comprising the G3-G5 domain of the human LAMA2 gene; or
d) a first domain comprising the heparin-binding domain of HBEGF and the EGF-like domain of HBEGF, and a second domain comprising the G3-G5 domain of the human LAMA2 gene.

For example, the disclosure also provides for use of a protein for the preparation of a medicament for treating a treating laminin-deficient muscular dystrophy in a patient in need thereof, wherein the protein comprises the amino acid sequence of SEQ ID NO: 19, SEQ ID NO: 20, SEQ ID NO: 21 or SEQ ID NO: 22.

Also provided are methods for treating a dystroglycanopathy comprising administering to a patient in need thereof a rAAV, wherein the genome of the rAAV comprises a polynucleotide encoding a protein comprising:
a) a first domain comprising the heparin-binding domain of HBEGF and a second domain comprising DAG1alpha or
b) a first domain comprising the heparin-binding domain of HBEGF and the EGF-like domain of HBEGF, and a second domain comprising DAG1alpha.

For example, the methods of treating a dystroglycanopathy comprising administering to a patient in need thereof any of the following: any of the polynucleotides disclosed herein which encode a protein comprising DAG1alpha as the second domain, any of the rAAV or rAAV particles disclosed herein which comprise a polynucleotide encoding a protein comprising DAG1alpha as the second domain.

Still further provided are methods for treating a dystroglycanopathy comprising administering to a patient in need thereof a protein comprising:
a) a first domain comprising the heparin-binding domain of HBEGF and a second domain comprising DAG1alpha or
b) a first domain comprising the heparin-binding domain of HBEGF and the EGF-like domain of HBEGF, and a second domain comprising DAG1alpha.

For example, the methods for treating a dystroglycanopathy comprising administering a protein to a patient in need thereof, wherein the protein comprises the amino acid sequence of SEQ ID NO: 23 or SEQ ID NO: 24.

Also provided are compositions for treating a dystroglycanopathy comprising a rAAV, wherein the genome of the rAAV comprises a polynucleotide encoding a protein comprising:
a) a first domain comprising the heparin-binding domain of HBEGF and a second domain comprising DAG1alpha or
b) a first domain comprising the heparin-binding domain of HBEGF and the EGF-like domain of HBEGF, and a second domain comprising DAG1alpha.

For example, the disclosure provides compositions for treating a dystroglycanopathy comprising any of the following: any of the polynucleotides disclosed herein which encode a protein comprising DAG1alpha as the second domain, or any of the rAAV or rAAV particles disclosed herein which comprise a polynucleotide encoding a protein comprising DAG1alpha as the second domain.

Still further provided are compositions for treating a dystroglycanopathy comprising a protein comprising:
a) a first domain comprising the heparin-binding domain of HBEGF and a second domain comprising DAG1alpha or
b) a first domain comprising the heparin-binding domain of HBEGF and the EGF-like domain of HBEGF, and a second domain comprising DAG1alpha.

For example, the compositions for treating a dystroglycanopathy comprise a protein wherein the protein comprises the amino acid sequence of SEQ ID NO: 23 or SEQ ID NO: 24.

Also provided is use of a rAAV for the preparation of a medicament for treating a dystroglycanopathy in a patient in need thereof, wherein the genome of the rAAV comprises a polynucleotide encoding a protein comprising:
a) a first domain comprising the heparin-binding domain of HBEGF and a second domain comprising DAG1alpha or
b) a first domain comprising the heparin-binding domain of HBEGF and the EGF-like domain of HBEGF, and a second domain comprising DAG1alpha.

For example, the disclosure provides for use of any of the following: any of the polynucleotides disclosed herein which encode a protein comprising DAG1alpha as the second domain or any of the rAAV or rAAV particles disclosed herein which comprise a polynucleotide encoding a protein comprising DAG1alpha as the second domain, for the preparation of a medicament for treating a dystroglycanopathy in a patient in need thereof.

Still further provided is a use of a protein for the preparation of a medicament for treating a dystroglycanopathy in a patient in need thereof, wherein the protein comprises:
a) a first domain comprising the heparin-binding domain of HBEGF and a second domain comprising DAG1alpha or
b) a first domain comprising the heparin-binding domain of HBEGF and the EGF-like domain of HBEGF, and a second domain comprising DAG1alpha.

For example, the disclosure provides a use of a protein for the preparation of a medicament for treating a dystroglycanopathy in a patient in need thereof, wherein the protein comprises the amino acid sequence of SEQ ID NO: 23 or SEQ ID NO: 24.

In methods, uses or composition for treating laminin-deficient muscular dystrophy provided, the laminin-deficient muscular dystrophy may be, for example, MDC1A.

In any of the methods, uses or compositions for treating a dystroglycanopathy, the dystroglycanopathy may be, for example, Walker Warburg syndrome, Muscle Eye Brain disease, Fukuyama Congenital Muscular Dystrophy, MDC1C, MDC1D, LGMD2I, LGMD2K, LGMD2M, LGMD2N, LGMD2O, LGMD2P, LGMD2T or LGMD2U.

Examples of the provided proteins are described in Table 1.

TABLE 1

| | | | Encoded by | | |
|---|---|---|---|---|---|
| | | | nucleotide in | Protein | Plasmid |
| Therapeutic | Linker/ | | FIG. or SEQ | SEQ | SEQ ID |
| protein | Laminin | FIG. | ID NO: | ID NO: | No:/FIG. |
| HB-LAMA2 | Ending after | 3 | 14 to 3235 of | 19 | SEQ ID |
| (G1-G5) | HB domain | | FIG. 3 SEQ | | NO: 2 |
| | | | ID NO: 1 | | FIG. 19 |
| HBEGF- | Complete | 4 | 14 to 3361 of | 20 | SEQ ID |
| LAMA2 | soluble form | | FIG. 4 SEQ | | NO: 4 |
| (G1-G5) | HBEGF | | ID NO: 3 | | FIG. 20 |
| HB-LAMA2 | Ending after | 5 | 14 to 1930 of | 21 | SEQ ID |
| (G3-G5) | HB domain | | FIG. 5 SEQ | | NO: 6 |
| | | | ID NO: 5 | | FIG. 21 |
| HBEGF- | Complete | 6 | 14 to 2056 of | 22 | SEQ ID |
| LAMA2 | soluble form | | FIG. 6 SEQ | | NO: 8 |
| (G3-G5) | HBEGF | | ID NO: 7 | | FIG. 22 |
| HB-DAG1 | Ending after | 7 | 14 to 1360 of | 23 | SEQ ID |
| | HB domain | | FIG. 7 SEQ | | NO: 10 |
| | | | ID NO: 9 | | FIG. 23 |
| HBEGF- | Complete | 8 | 14-1486 of | 24 | SEQ ID |
| DAG1 | soluble form | | FIG. 8 SEQ | | NO: 12 |
| | HBEGF | | ID NO: 11 | | FIG. 24 |

### BRIEF DESCRIPTION OF THE DRAWINGS

The patent or application file contains at least one drawing executed in color. Copies of this patent or patent application publication with color drawings and color photographs will be provided by the Office upon request and payment of the necessary fee.

FIG. 1A depicts the dystrophin-associated glycoprotein (DAG) complex. FIG. 1B shows α dystroglycan is not only abnormally glycosylated in dystroglycanopathies, which removes its normal laminin binding function, but α dystroglycan protein is reduced in diseased muscles. FIG. 1C shows therapeutic proteins described herein will allow α dystroglycan to link to the muscle membrane by binding to β dystroglycan, which is present in normal amounts, and link to the ECM, even without its proper ECM-binding glycans, via binding of HBEGF to heparin sulfate proteoglycans of the ECM. This will reconstitute the lost linkages of α dystroglycan to the ECM and to the muscle membrane. Use of methods described herein providing these therapeutic proteins is indicated for treatment of all 18-plus genetic forms of dystroglycanopathies, making the methods powerful alternatives to gene replacement strategies in which each dystroglycanopathy would require development of a different gene therapy.

FIG. 2A depicts MDC1A is caused by loss of function mutations in the LAMA2 gene, which encodes laminin α2, an extracellular matrix (ECM) protein that surrounds each muscle cell in the body. LAMA2 is required for muscle cell adherence to the ECM and for muscle membrane stability. FIG. 2B shows therapeutic proteins described herein can anchor the LAMA2 G1-G5 domains to the ECM where the LAMA2 G1-G5 domains would normally be present, so the LAMA2 G1-G5 domains can function as they do in native laminin α2. Use of methods described herein providing these therapeutic proteins is thus indicated for MDC1A.

FIG. 3 shows the polynucleotide sequence encoding a therapeutic protein HB-EGF (ending at heparin binding domain)-LAMA2 G1-G5. The therapeutic protein is encoded by nucleotides of the invention comprises nucleotides 14 to 3235, which also correspond to SEQ ID NO: 1.

FIG. 4 shows the polynucleotide sequence encoding a therapeutic protein HB-EGF (complete soluble form)-LAMA2 G1-G5. The therapeutic protein is encoded by nucleotides of the invention comprises nucleotides 14 to 3361, which also correspond to SEQ ID NO: 3.

FIG. 5 shows the polynucleotide sequence encoding a therapeutic protein HB-EGF (ending at heparin binding domain)-LAMA2 G3-G5. The therapeutic protein is encoded by nucleotides of the invention comprises nucleotides 14 to 1930, which also correspond to SEQ ID NO: 5.

FIG. 6 shows the polynucleotide sequence encoding a therapeutic protein HB-EGF (complete soluble form)-LAMA2 G3-G5. The therapeutic protein is encoded by nucleotides of the invention comprises nucleotides 14 to 2056, which also correspond to SEQ ID NO: 7.

FIG. 7 shows the polynucleotide sequence encoding a therapeutic protein HB-EGF (ending at heparin binding domain)-DAG1 (native processed alpha DG gene). The therapeutic protein is encoded by nucleotides of the invention comprises nucleotides 14 to 1360, which also correspond to SEQ ID NO: 9.

FIG. 8 shows the polynucleotide sequence encoding a therapeutic protein HB-EGF (complete soluble form)-DAG1 (native processed alpha DG gene). The therapeutic protein is encoded by nucleotides of the invention comprises nucleotides 14 to 1486, which also correspond to SEQ ID NO: 11.

FIG. 9 shows expression of therapeutic proteins described herein by recombinant mammalian host cells.

FIG. 10 shows that sHB-EGF can be secreted from muscles and stick to the extracellular matrix.

FIG. 11 shows that sHB-EGF induces expression of therapeutic surrogate muscular dystrophy genes. Full length HBEGF does not induce therapeutic gene expression.

FIG. 12 shows that sHB-EGF induces Akt tyrosine kinase cascade in skeletal muscle and can stimulate muscle growth and regeneration.

FIG. 13 shows an exemplary rAAV genome encoding the therapeutic protein HB-EGF (complete soluble form)-LAMA2 G1-G5.

FIG. 14 shows an exemplary rAAV genome encoding the therapeutic protein HB-EGF (complete soluble form)-DAG1 (native processed alpha DG gene).

FIG. 15 provides immunohistochemical staining for HB-EGF and LG5 (denoted in figure as 4H8-2) after intramuscular injection of rAAV9.CMV vectors containing HBEGF, HBEGF.LAMA2(G1-G5), HBEGF.LAMA2(G3-G5), HB.LAMA2(G1-G5), HB.LAMA2(G3-G5), or LAMA2(G1-G5) in wild type mice. Mock injected mice (buffer alone) are shown as a negative control. 4H8-2 is an anti-laminin antibody to show muscle cells in the sections.

FIG. 16 provides immunohistochemistry staining for HB-EGF and Laminin Globular Domain (LG5) in muscles injected IM with rAAV9.HBEGF-LAMA2(G1-G5), HB-LAMA2(G1-G5), or LAMA2(G1-G5). The lower panels below show staining for secondary antibody alone.

FIG. 17 is a graph demonstrating that rAAV9.CMV.HB.LAMA(G1-G5) prevented loss of muscle strength in dy/dy mice. Mice were injected IV with 1×10¹² vg of rAAV9.CMV vectors containing HBEGF.LAMA2(G1-G5), HB.LAMA2(G1-G5), or HB.LAMA2(G3-G5). Mice were compared to mock-injected dy/dy disease controls and wild type normal controls at 2 months and 3 months post-injection. Mixed (50:50) female:male genders were used in all groups. Errors are SEM for n=12 (wild type and dy/dy mock), 6 (SHB-EGF.LAMA2G1-G5 and HB.LAMA2G1-G5) or 5 (HB.LAMA2G3-G5) animals per group, with five measures averaged per data point. * p<0.05, ** p<0.01, *** p<0.001

FIG. 18 provides immunohistochemistry staining for HB-EGF and LG5 to demonstrate expression of HB.LAMA2(G1-G5) in dy/dy muscle (triceps) at 4 months of age after IV injection at P1. Muscle sections from the triceps muscle of 4-month old wild type and dy/dy mice, either mock-injected or injected with 1×10¹² vg rAAV9.CMV.HB-LAMA2(G1-G5) were stained with antibodies specific to HBEGF (green), to recognize transgenic protein, and to collagen IV (Col(IV), red), to recognize all muscle cells. DAPI is added in blue to stain nuclei. Merged tricolor images are shown.

FIG. 19 provides the plasmid sequence of pAAV.CMV.HB.LAMA1(G1-G5) (SEQ ID NO: 2), the rAAV genome corresponds to nucleotides 3590 to 8215.

FIG. 20 provides the plasmid sequence of pAAV.CMV.HBEGF LAMA2(G1-G5) (SEQ ID NO:4), the rAAV genome corresponds to nucleotides 3590-8341.

FIG. 21 provides the plasmid sequence of pAAV.CMV. HB LAMA2 (G3-G5) (SEQ ID NO: 6), the rAAV genome corresponds to nucleotides 36909-6929.

FIG. 22 provides the plasmid sequence of pAAV.CMV.HBEGF.LAMA2 (G3-G5) (SEQ ID NO: 8), the rAAV genome corresponds to nucleotides 3590-7036.

FIG. 23 provides the plasmid sequence of pAAV.CMV. HB.DAG1 (alpha) (SEQ ID NO: 10), the rAAV genome corresponds to nucleotides, the rAAV genome corresponds to nucleotides 3590 to 6340.

FIG. 24 provides the plasmid sequence of pAAV.CMV.HB.DAG1(alpha) (SEQ ID NO: 13), the rAAV genome corresponds to nucleotides 3590-6049.

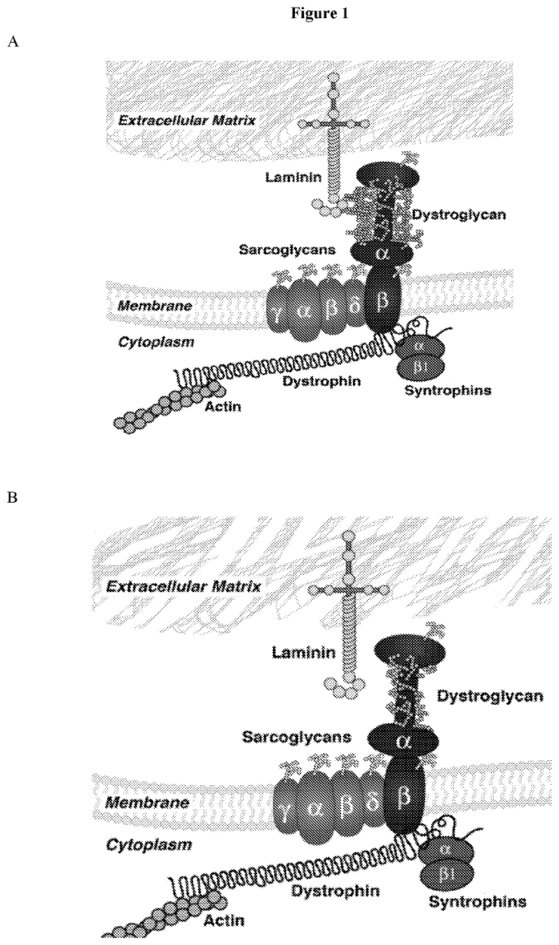

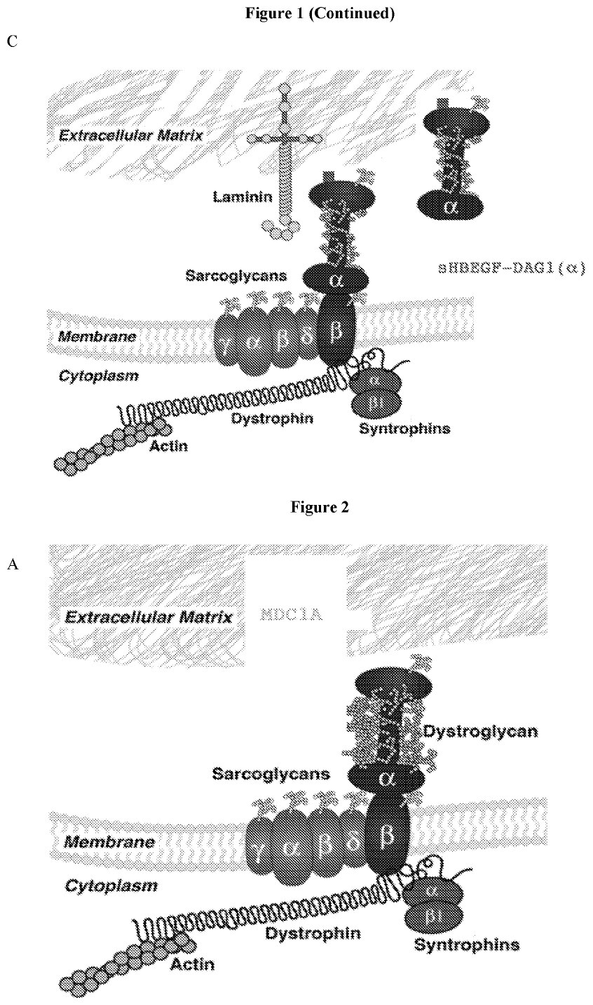

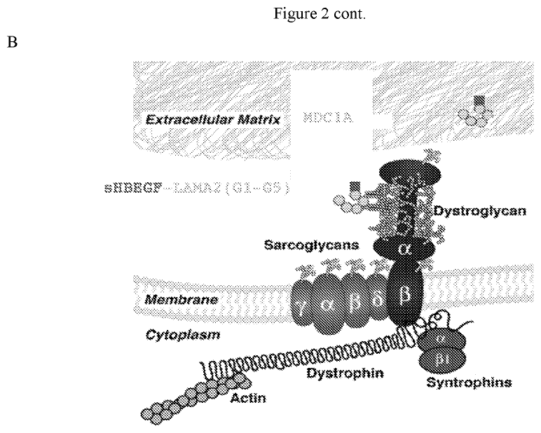

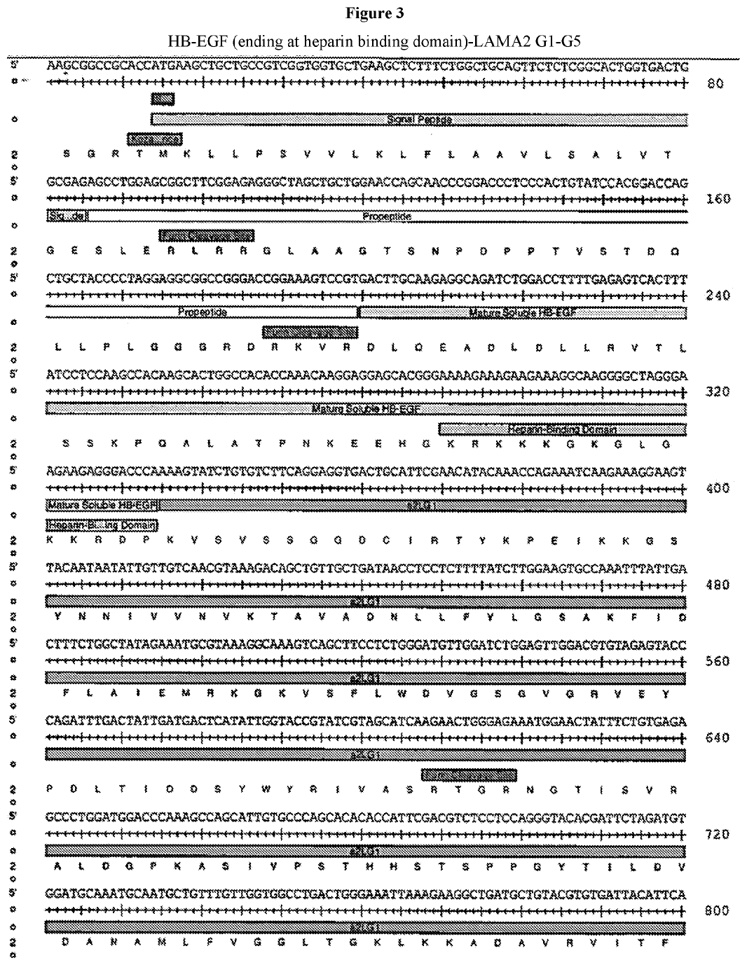

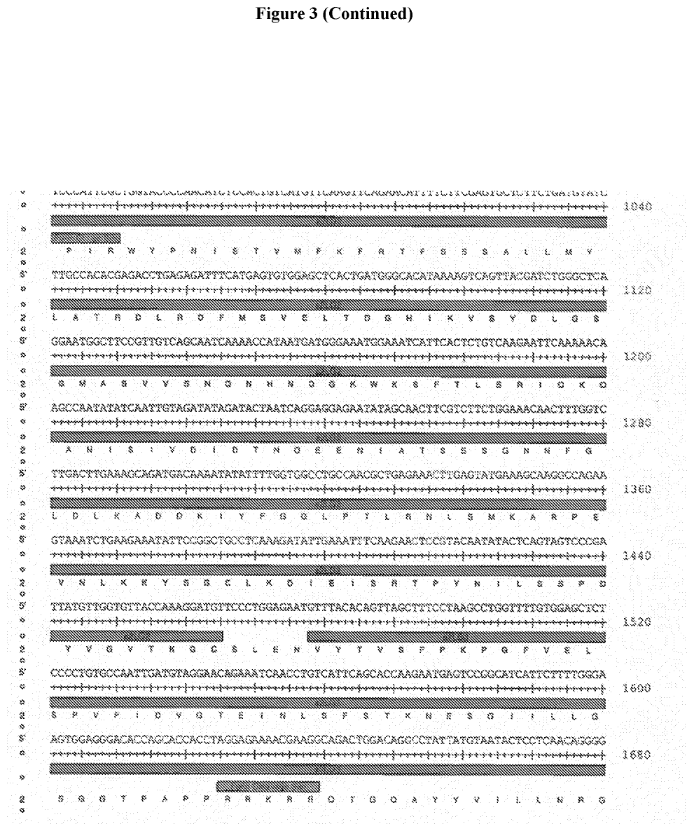

### DETAILED DESCRIPTION

Methods and products are provided herein for treatment of dystroglycanopathies (including, but not limited to, Walker Warburg syndrome, Muscle Eye Brain disease, Fukuyama Congenital Muscular Dystrophy, MDC1C, MDC1D, LGMD2I, LGMD2K, LGMD2M, LGMD2N, LGMD2O, LGMD2P, LGMD2T and LGMD2U) and laminin-deficient muscular dystrophies (including, but not limited to, MDC1A) which utilize the lysine-rich heparin-binding domain of HBEGF. Heparin sulfate proteoglycans are abundant in the extracellular matrix (ECM) and, as shown herein, the overexpression of HBEGF in muscle leads to localization of HBEGF in the muscle ECM. In methods described herein, the membrane anchoring defects in dystroglycanopathies and laminin-deficient muscular dystrophies are treated using the heparin-binding domain of HBEGF as a “linker” domain in therapeutic proteins.

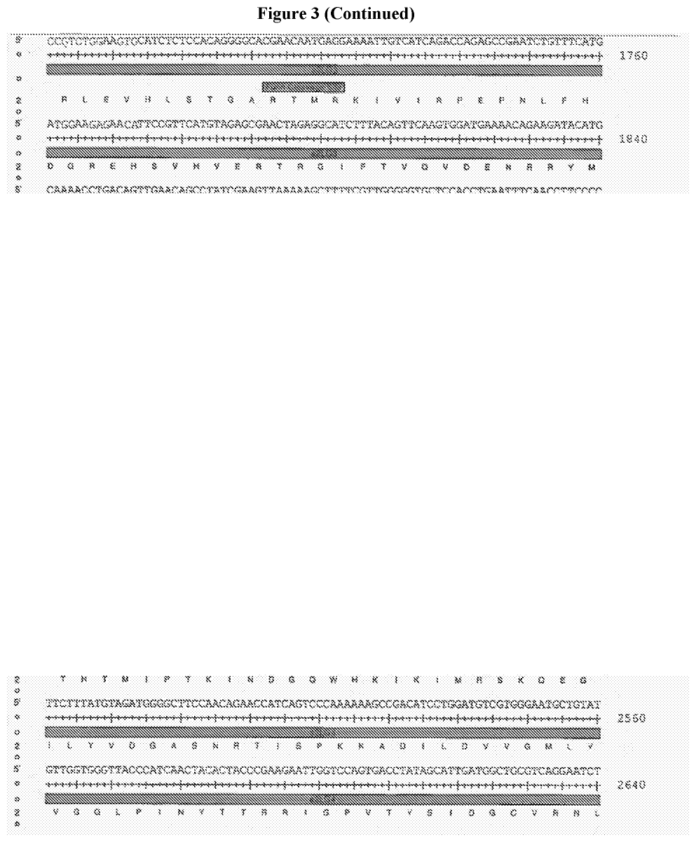

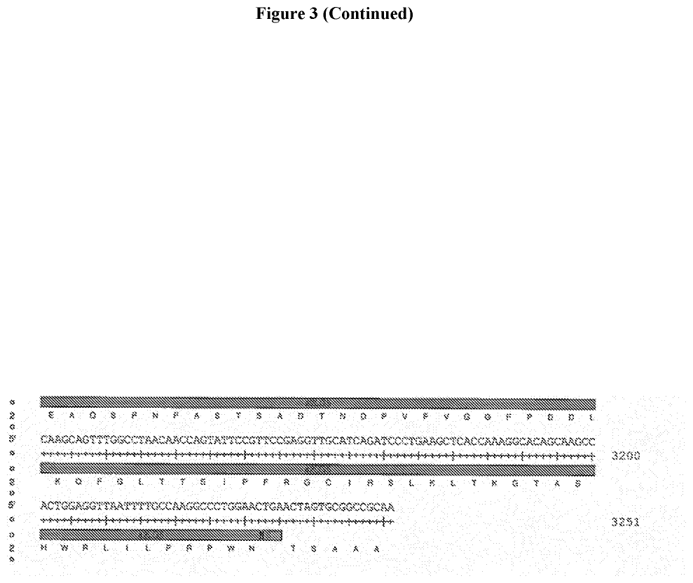

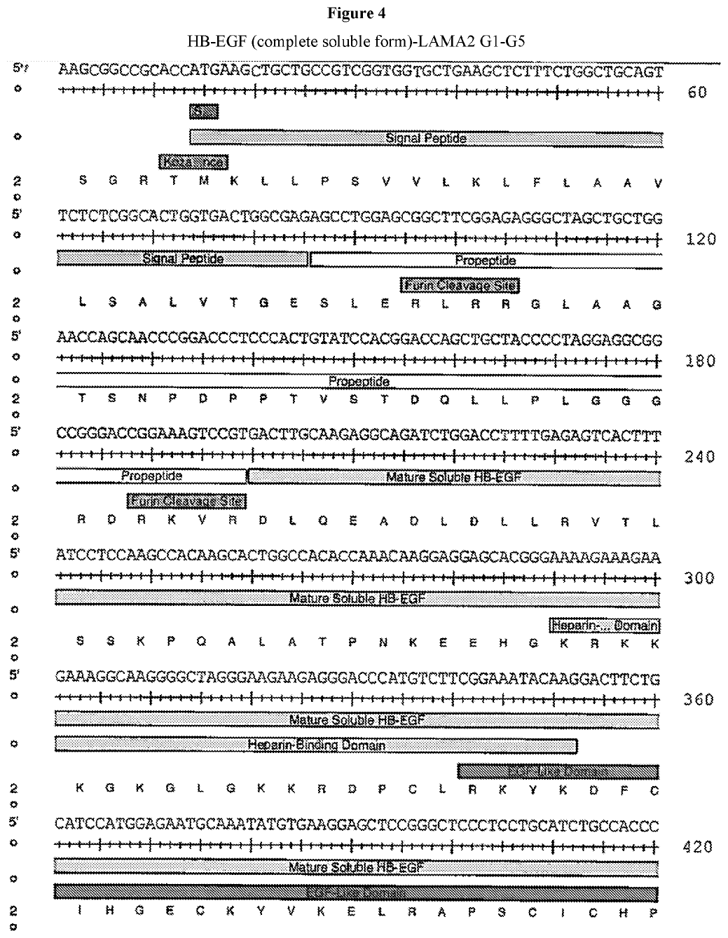

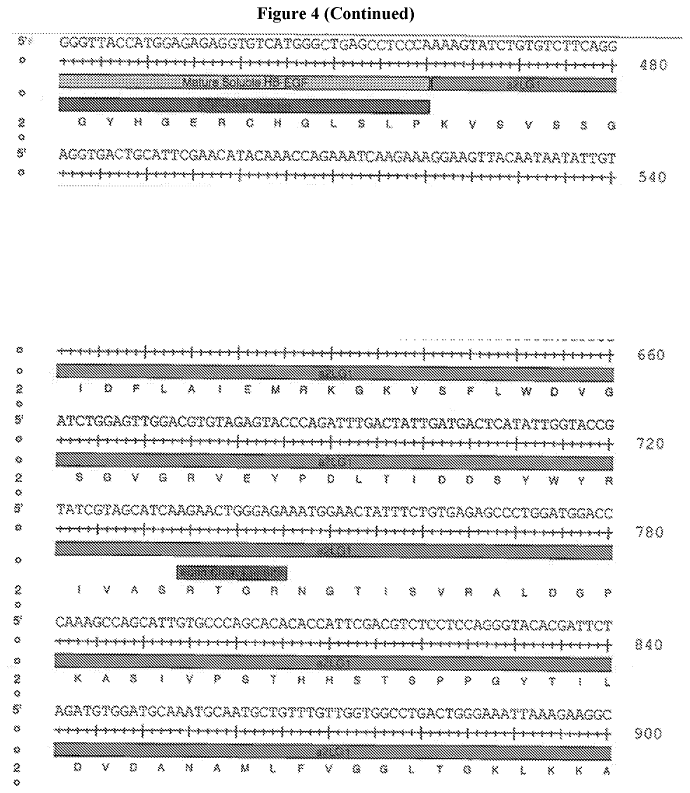

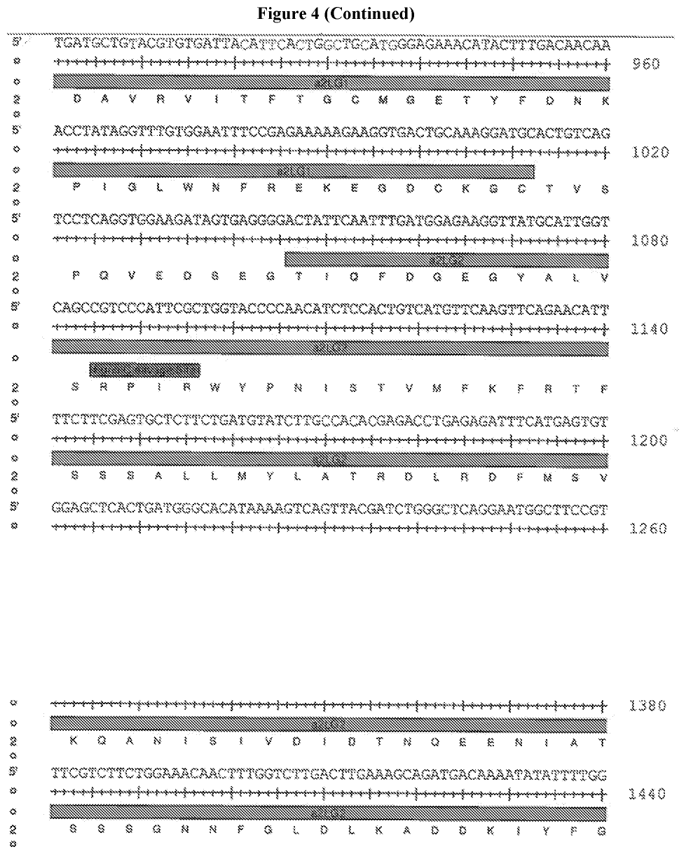

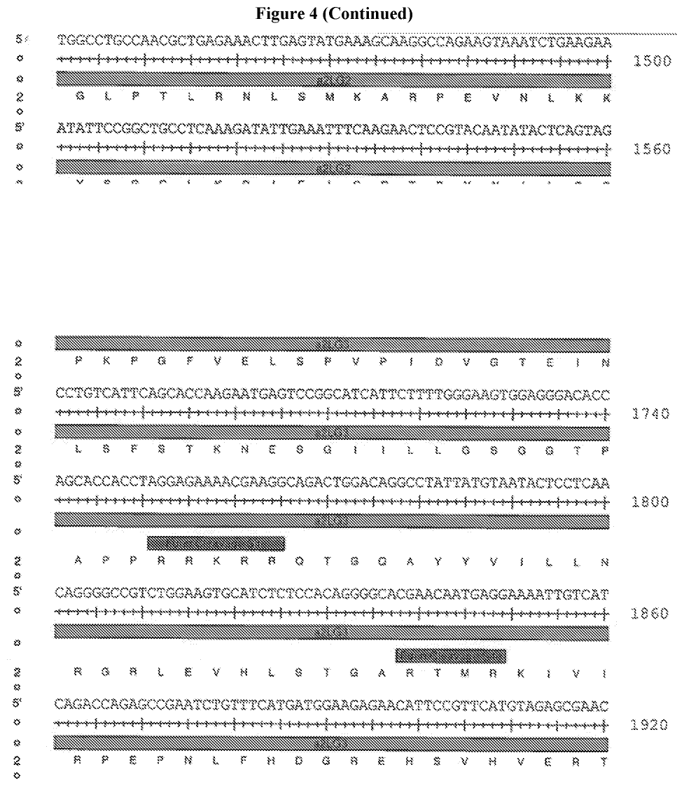

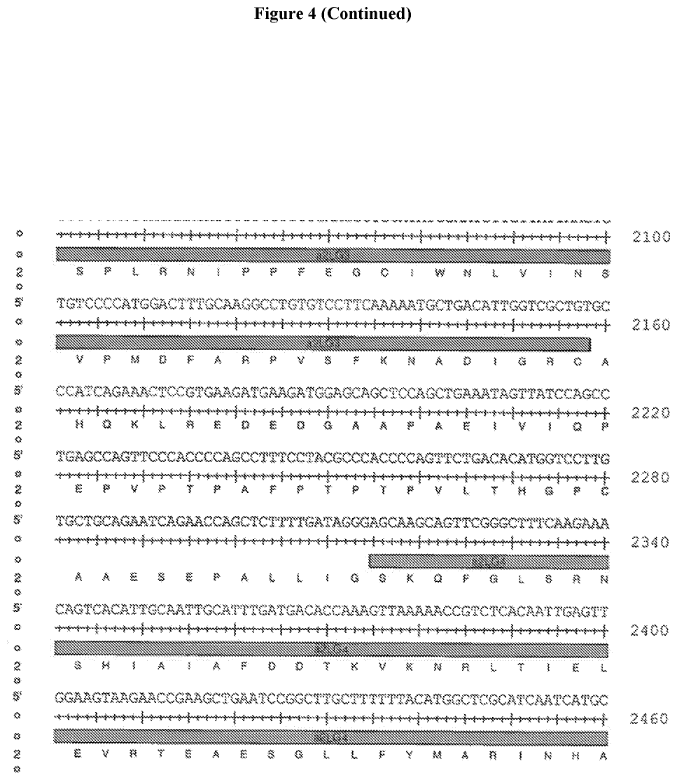

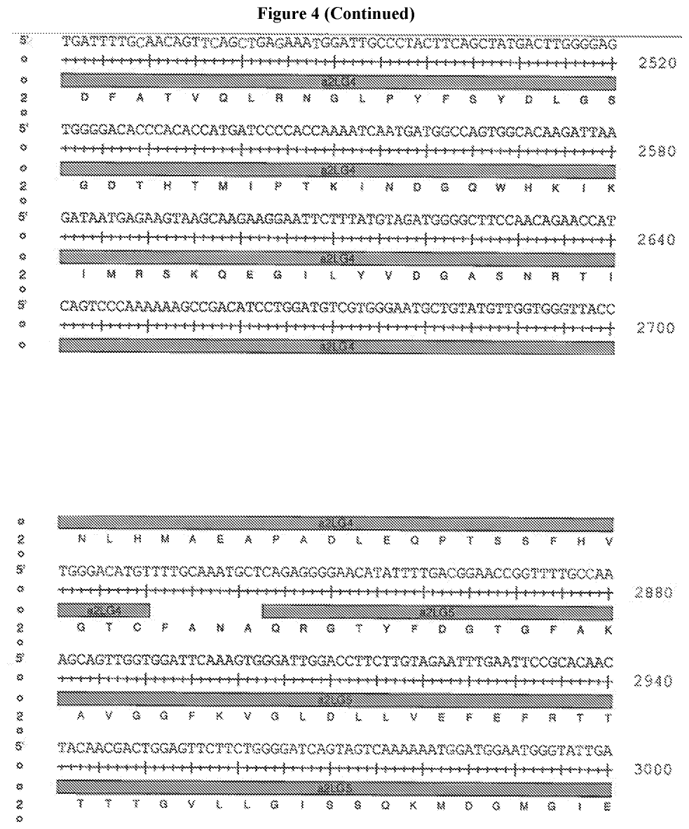

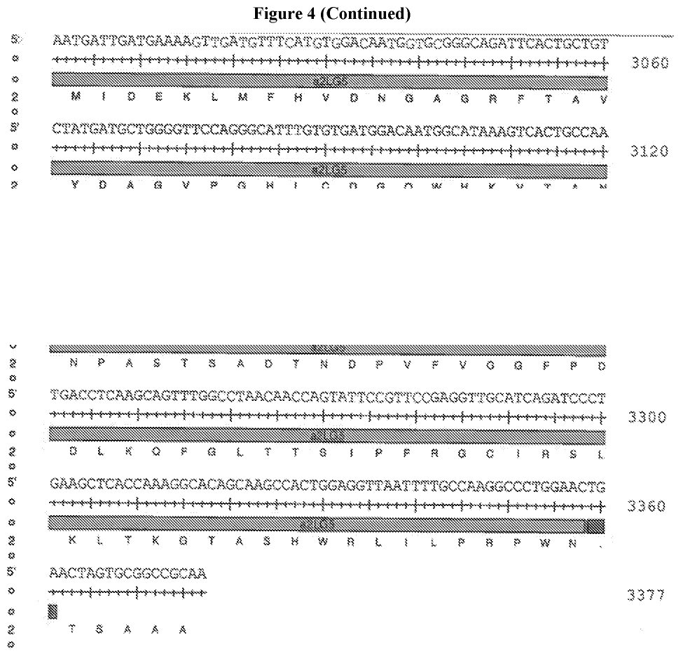

Here, the term “HBEGF” refers to the entire HBEGF sequence up to and including the bioactive EGF domain, but lacking the transmembrane domain, which thereby allows HBEGF secretion (FIG. 4). The HBEGF fragment contains four domains from the HBEGF gene, the signal peptide, which allows entry into the secretory pathway, the prepro-peptide, which allows folding and stabilization of the protein, the heparin binding domain, which allows for increased interaction with the extracellular matrix, and the bioactive EGF domain, which allows for HBEGF signaling. In the proteins disclosed herein, the coding sequence for these domains are then then linked to laminin alpha 2 or dystroglycan coding sequences. A second “HB” fragment is also used (FIG. 3). The HB fragment only contains 3 of the four domains found in HBEGF: the signal peptide, the pre-pro-peptide, and the heparin binding domain (and so HB lacks the bioactive EGF domain). When linked to laminin alpha 2 or dystroglycan protein fragments, the HB domain allows for increased association with the ECM but without increasing EGF or HBEGF signaling.

The HBEGF or HB linker domain targets a protein to the extracellular matrix of a cell and acts to anchor this protein to the extracellular domain of a cell, such as a muscle cell. Polynucleotides encoding therapeutic proteins are delivered to a patient (for example, delivery by a recombinant AAV encoding the therapeutic proteins), or the therapeutic proteins are delivered to a patient.

For example, for all of the dystroglycanopathies, a coding sequence for HBEGF heparin-binding domain is fused to a coding sequence for α-dystroglycan, creating a polynucleotide encoding the therapeutic protein HBEGF-DAG1(α). In addition to α-dystroglycan hypoglycosylation, α-dystroglycan protein levels are reduced in dystroglycanopathies. In methods described herein, the HBEGF domain of HBEGF-DAG1(α) binds the ECM heparin sulfate proteoglycans, while the α-dystroglycan domain binds β-dystroglycan, linking the sarcolemma to the ECM despite hypoglycosylation in the dystroglycanopathies. Four examples of such HBEGF-DAG1(α) therapeutic proteins are:

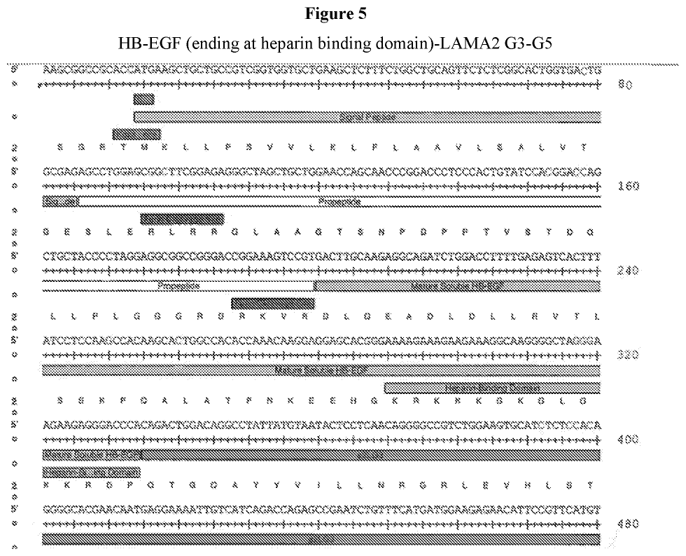

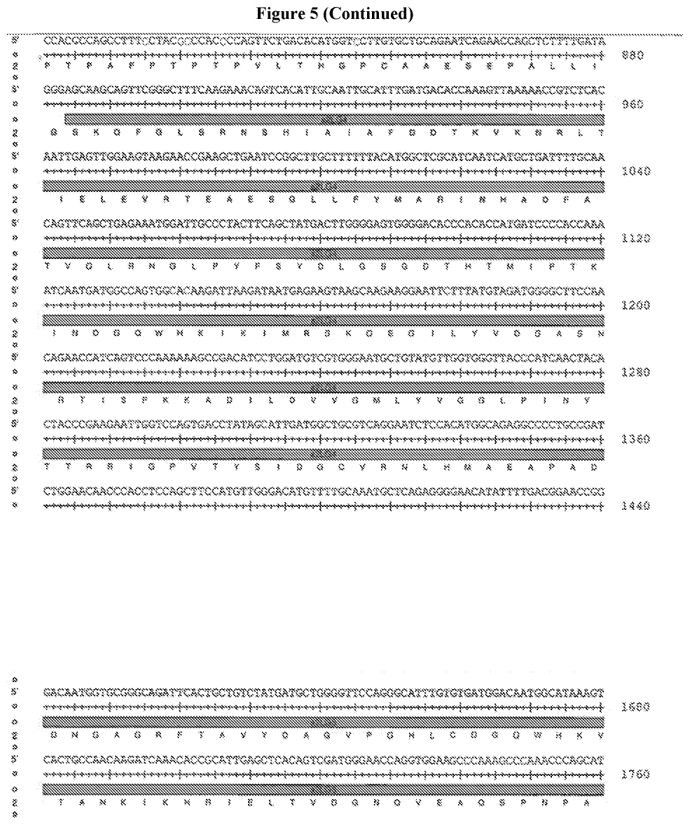

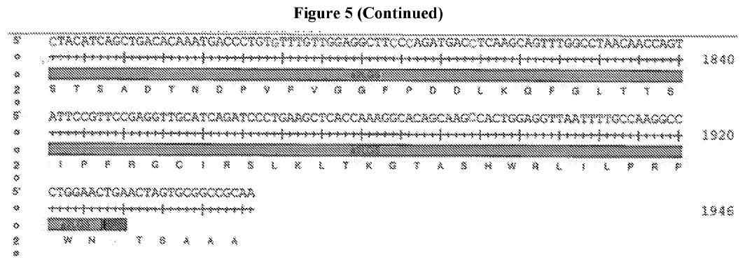

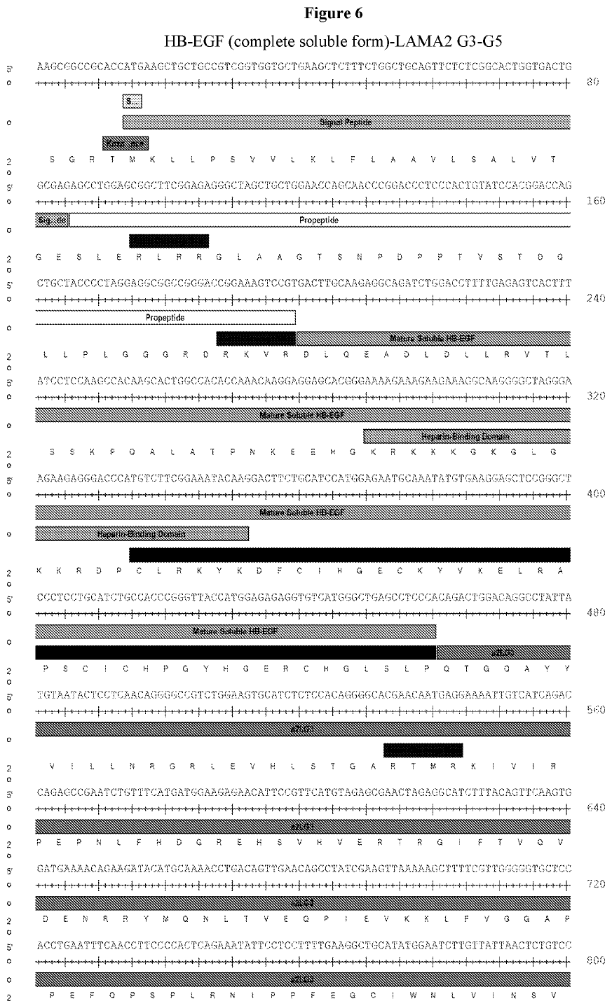

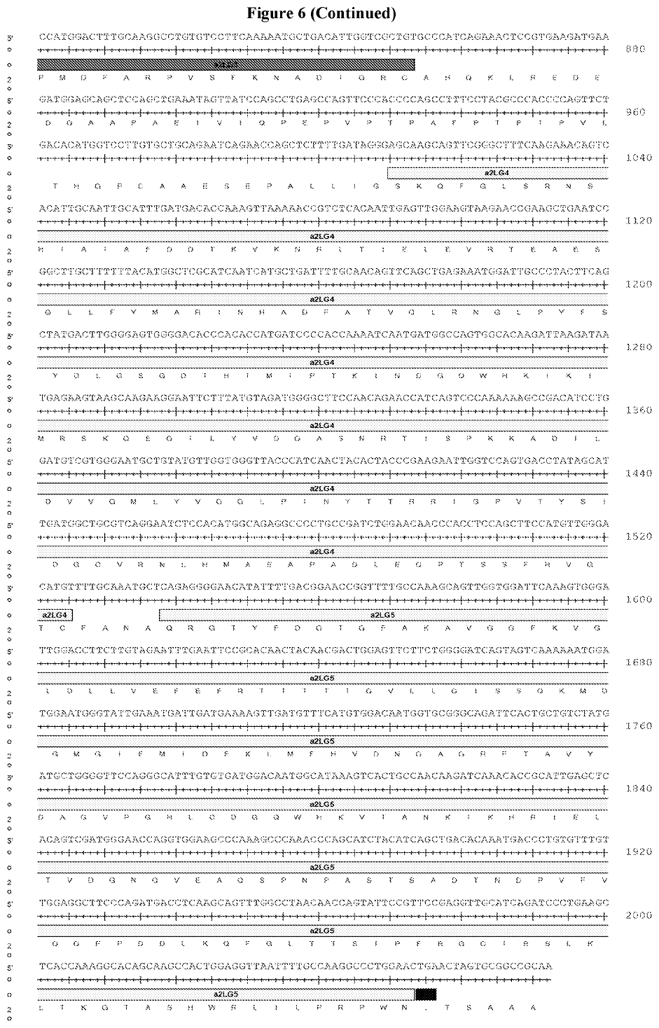

HB-EGF (ending at heparin binding domain)-LAMA2 G1-G5 (encoded by the polynucleotide of FIG. 3),
HB-EGF (complete soluble form)-LAMA2 G1-G5 (encoded by the polynucleotide of FIG. 4),
HB-EGF (ending at heparin binding domain)-LAMA2 G3-G5 (encoded by the polynucleotide of FIG. 5), and
HB-EGF (complete soluble form)-LAMA2 G3-G5 (encoded by the polynucleotide of FIG. 6).

The term “complete soluble form” herein indicates the therapeutic protein comprises the HBEGF heparin-binding and EGF-like domains, but not the transmembrane portion of HBEGF. The combination of the HBEGF heparin-binding and EGF-like domains of HBEGF corresponds to the cleaved, active, soluble isoform of HBEGF. This term is referred to as ‘HBEGF” herein.

The term “ending at heparin binding domain” herein indicates that that therapeutic protein comprises only HBEGF heparin-binding domain and does not comprise the EGF-like domain or the transmembrane portion of HBEGF. This term is abbreviated as ‘HB” herein.

For example, for laminin-deficient muscular dystrophies such as MDC1A, a coding sequence for HBEGF heparin-binding domain is fused to a coding sequence for the globular (G) domains 1-5 of laminin-α2, creating a polynucleotide encoding the therapeutic protein HBEGF-LAMA2(G1-5). The laminin-α2 G domains bind glycosylated α-dystroglycan and also integrins at the sarcolemma, and are encoded by a part of the LAMA2 gene. In methods described herein, the HBEGF domain of HBEGF-LAMA2(G1-5) binds the ECM heparin sulfate proteoglycans, while the G domains bind α-dystroglycan, linking the sarcolemma to the ECM despite the absence of full-length laminin-α2 in MDC1A. Two examples of such HBEGF-LAMA2(G1-5) therapeutic proteins are:

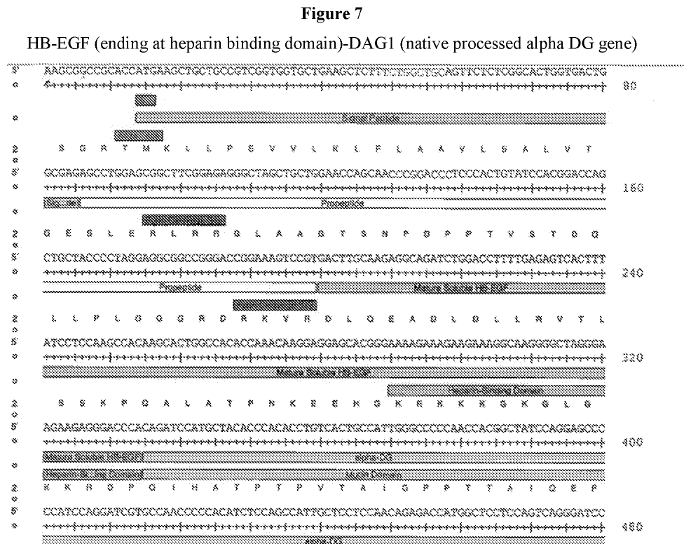

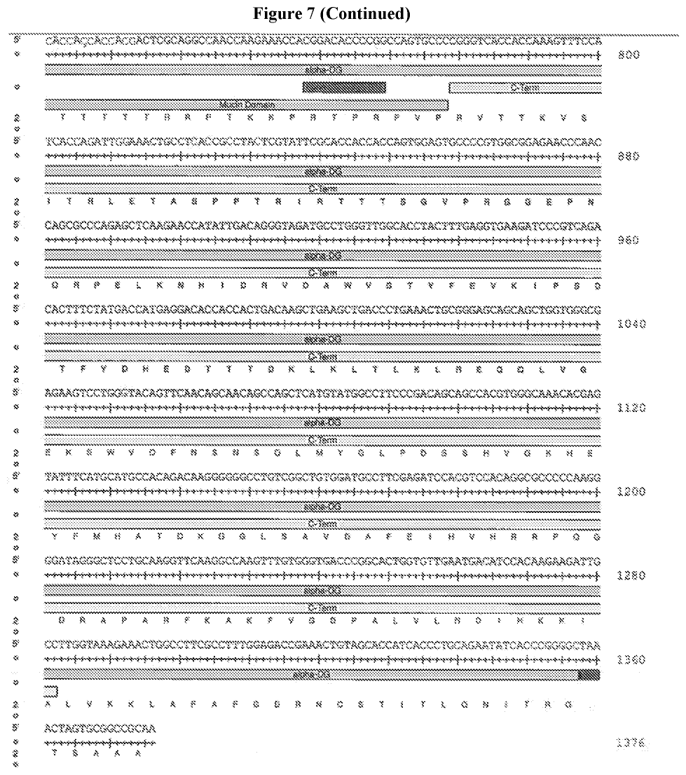

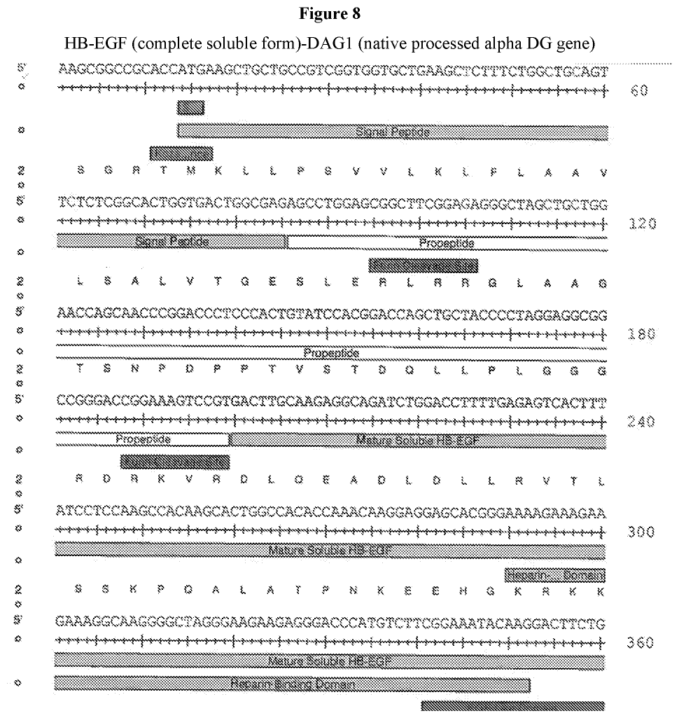

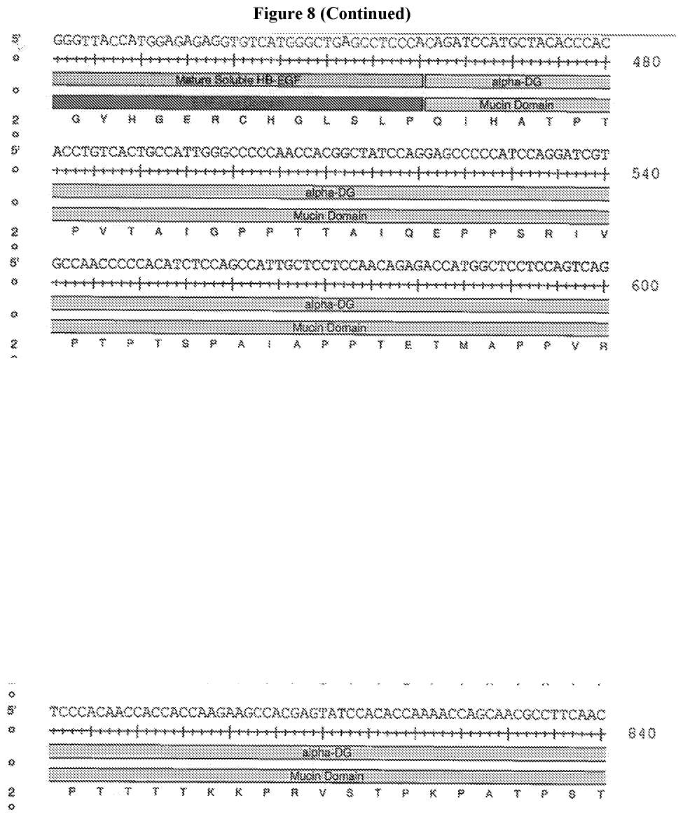

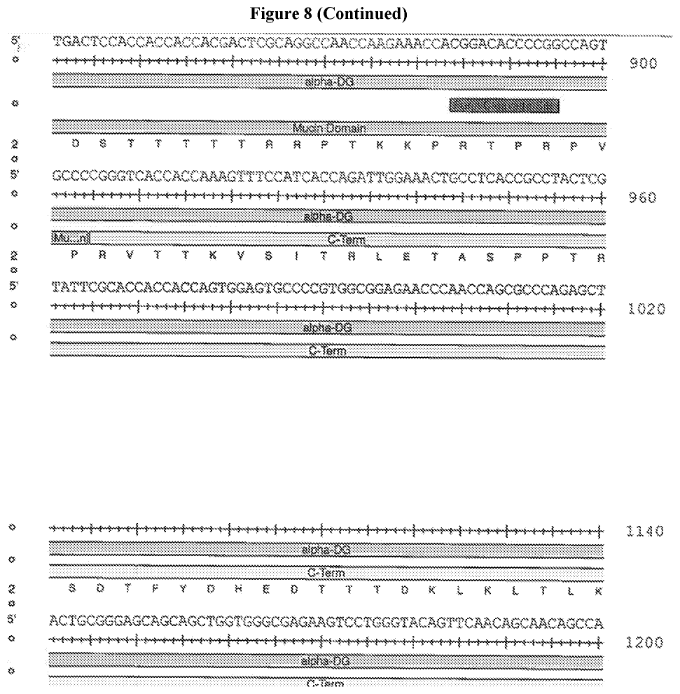

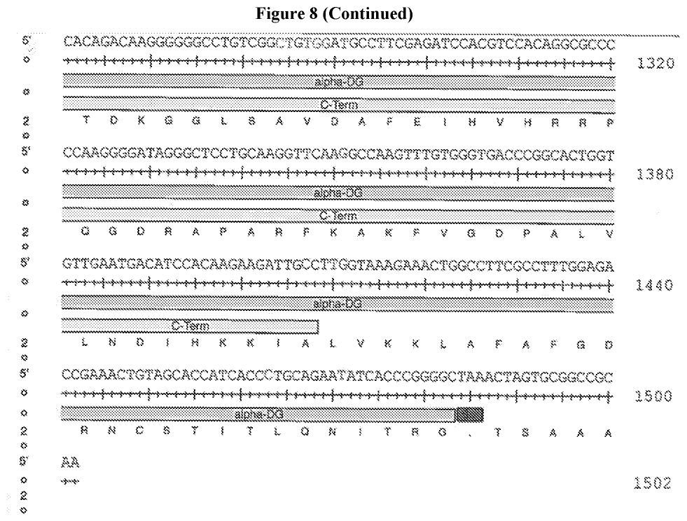

HB-EGF (ending at heparin binding domain)-DAG1 (native processed alpha DG gene) (encoded by the polynucleotide of FIG. 7) and
HB-EGF (complete soluble form)-DAG1 (native processed alpha DG gene) (encoded by the polynucleotide of FIG. 8).

Furthermore, both dystroglycanopathies and laminin-deficient muscular dystrophies (such as MDC1A) are associated with reduced muscle regeneration and, in embodiments of methods described herein wherein the therapeutic proteins comprise a HBEGF heparin-binding domain and HBEGF EGF-like domain, patients also benefit from trophic signaling of the HBEGF EGF-like domain of the therapeutic proteins which results in the alteration of expression of genes including Pax7, MyoD), Myogenin and Myh3 increasing myogenesis and muscle regeneration.

Thus, polynucleotides are provided encoding the therapeutic proteins. Embodiments include a polynucleotide comprising the polynucleotide sequence set forth in FIG. 3, 4, 5, 6, 7 or 8. Other embodiments include a polynucleotide encoding the same amino acid sequence as the polynucleotide sequence set forth in FIG. 3, 4, 5, 6, 7 or 8. Still other embodiments include a polynucleotide comprising a polynucleotide consisting of the polynucleotide sequence set forth in FIG. 3, 4, 5, 6, 7 or 8.

In one embodiment, the provided polynucleotides comprise one of the following: i) the nucleotide sequence set forth in FIG. 3, ii) a nucleotide sequence comprising nucleotides 14 to 3235 set out in FIG. 3, iii) the nucleotide sequence of SEQ ID NO: 1, or iv) a nucleotide sequence encoding the amino acid sequence of SEQ ID NO: 19.

In another embodiment, the provided polynucleotides comprise one of the following: i) the nucleotide sequence set forth in FIG. 4, ii) a nucleotide sequence comprising nucleotides 14 to 3361 set forth in FIG. 4, iii) the nucleotide sequence of SEQ ID NO: 3 or iv) a nucleotide sequence encoding the amino acid sequence of SEQ ID NO: 20.

In a further embodiment, the provided polynucleotides comprise one of the following: i) the nucleotide sequence set forth in FIG. 5, ii) a nucleotide sequence comprising nucleotides 14 to 1930 set forth in FIG. 5, iii) the nucleotide sequence of SEQ ID NO: 5 or iv) a nucleotide sequence encoding the amino acid sequence of SEQ ID NO: 21.

In another embodiment, the provided polynucleotides comprise one of the following: i) the nucleotide sequence set forth in FIG. 6, ii) a nucleotide sequence comprising nucleotides 14 to 2056 set forth in FIG. 6, iii) the nucleotide sequence of SEQ ID NO: 7 or iv) a nucleotide sequence encoding the amino acid sequence of SEQ ID NO: 22.

In an embodiment, the provided polynucleotides comprise one of the following: i) the nucleotide sequence set forth in FIG. 7, ii) a nucleotide sequence comprising nucleotides 14 to 1360 set forth in FIG. 7, iii) the nucleotide sequence of SEQ ID NO: 9 or iv) a nucleotide sequence encoding the amino acid sequence of SEQ ID NO: 23.

In another embodiment, the provided polynucleotides comprise one of the following: i) the nucleotide sequence set forth in FIG. 8, ii) a nucleotide sequence comprising nucleotides 14-1486 set forth in FIG. 8, iii) the nucleotide sequence of SEQ ID NO: 11 or iv) a nucleotide sequence encoding the amino acid sequence of SEQ ID NO: 24.

Other polynucleotides provided include, but are not limited to, a polynucleotide that encodes an amino acid variant of a therapeutic polypeptide which retains the binding activity of the therapeutic protein, which polynucleotide has a nucleotide sequence at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the protein-coding nucleotide sequence set out in FIG. 3, 4, 5, 6, 7 or 8 or the nucleotide sequence of any of the provided polynucleotides.

Also provided herein are polynucleotides that encode an amino acid variant of a therapeutic polypeptide which retains the binding activity of the therapeutic protein, which polynucleotide hybridizes under stringent conditions to the protein-coding nucleotide sequence set out in FIG. 3, 4, 5, 6, 7 or 8, or the complement thereof or the nucleotide sequence of any of the provided polynucleotides. The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2^{nd} Ed., Cold Spring Harbor Laboratory, (Cold Spring Harbor, N.Y. 1989).

“Retains the binding activity” is contemplated herein to mean that the amino acid variant of the therapeutic protein encoded by a polynucleotide competes for binding with a therapeutic protein encoded by the nucleotide sequence set out in FIG. 3, 4, 5, or 6 to heparin sulfate proteoglycans and β-dystroglycan; or for binding with a therapeutic protein encoded by the nucleotide sequence set out in FIG. 7 or 8 to heparin sulfate proteoglycans and α-dystroglycan; or a therapeutic protein comprising the amino acid sequence of SEQ ID NO: 19, SEQ ID NO: 20, SEQ ID NO: 21SEQ ID NO: 22, SEQ ID NO: 23 OR SEQ ID NO: 24 to heparin sulfate proteoglycans and α-dystroglycan.

Recombinant expression vectors comprising one or more of the polynucleotides described herein are also provided. Recombinant AAV genomes comprising a polynucleotide described herein are also provided.

In expression vectors or recombinant AAV genomes described herein, the polynucleotide encoding the therapeutic protein is operatively linked to transcriptional control elements (including, but not limited to, promoters, enhancers and/or introns), specifically transcriptional control elements functional in target cells of interest. For example, suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). Other suitable mammalian promoters include heterologous mammalian promoters, for example, heat-shock promoters and the actin promoter. Also for example, AAV delivery methods may comprise transducing muscle or liver cells using muscle-specific transcriptional control elements, including, but not limited to, those derived from the actin and myosin gene families, such as from the myoD gene family [See Weintraub et al., Science, 251: 761-766 (1991)], the myocyte-specific enhancer binding factor MEF-2 [Cserjesi and Olson, Mol Cell Biol, 11: 4854-4862 (1991)], control elements derived from the human skeletal actin gene [Muscat et al., Mol Cell Biol, 7: 4089-4099 (1987)], muscle creatine kinase sequence elements [See Johnson et al., Mol Cell Biol, 9:3393-3399 (1989)] and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors [Semenza et al., Proc Natl Acad Sci USA, 88: 5680-5684 (1991)], steroid-inducible elements and promoters including the glucocorticoid response element (GRE) [Mader and White, Proc. Natl. Acad. Sci. USA, 90: 5603-5607 (1993)], the tMCK promoter [see Wang et al., Gene Therapy, 15: 1489-1499 (2008)], hybrid α-myosin heavy chain enhancer-/MCK enhancer-promoter (MHCK7) promoter [Salva et al. Mol Ther, 15: 320-329 (2007), the CK6 promoter [see Wang et al., supra] and other control elements. Thus, one example of a muscle-specific transcriptional control element is the tMCK promoter. An example of a liver-specific promoter is LSP [Wang and Verma, Proc. Natl. Acad. Sci. USA, 96, 3906-3910 (1999)]. As another promoter example, for production of the therapeutic proteins in recombinant host cells, the promoter may be a constitutive promoter such as the cytomegalous virus (CMV) promoter. Another example is LAMA2.

For the expression of therapeutic proteins described herein, provided are expression systems and constructs in the form of plasmids, expression vectors, transcription or expression cassettes that comprise at least one polynucleotide as described herein are also provided, as well host cells comprising such expression systems or constructs. As used herein, “vector” means any molecule or entity (e.g., polynucleotide, plasmid, bacteriophage or virus) suitable for use to transfer protein coding information into a host cell. Examples of vectors include, but are not limited to, plasmids, viral vectors, non-episomal mammalian vectors and expression vectors, for example, recombinant expression vectors. Expression vectors, such as recombinant expression vectors, are useful for transformation of a host cell.

Host cells are provided into which an expression vector, such as a recombinant expression vector, has been introduced. A host cell can be any prokaryotic cell (for example, E. coli) or eukaryotic cell (for example, yeast, insect, or mammalian cells (e.g., CHO cells)). Expression vectors can be introduced into prokaryotic or eukaryotic cells via conventional transformation or transfection techniques. For stable transfection of mammalian cells, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced polynucleotide can be identified by drug selection, among other methods. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into nuclei.

The method selected will in part be a function of the type of host cell to be used. These methods and other suitable methods are well known to the skilled artisan, and are set forth, for example, in Sambrook et al., Molecular Cloning: A Laboratory Manual, 3rd ed., Cold Spring Harbor Laboratory Press, Cold Spring Harbor, N.Y. (2001).

A host cell, when cultured under appropriate conditions, synthesizes protein that can be subsequently collected from the culture medium (if the host cell secretes it into the medium) or directly from the host cell producing it (if it is not soluble). The selection of an appropriate host cell will depend upon various factors, such as desired expression levels, polypeptide modifications that are desirable or necessary for activity (such as glycosylation or phosphorylation) and ease of folding into a biologically active molecule. As one example, Chinese hamster ovary cells overexpressing LARGE (CHO-LARGE cells) [Yoon et al., A Method to Produce and Purify Full-Length Recombinant Alpha Dystroglycan: Analysis of N-and O-Linked Monosaccharide Composition in CHO Cells with or without LARGE Overexpression, PLOS Curr. (2013 January 2)] are contemplated for use in producing glycosylated therapeutic proteins described herein.

Mammalian cell lines available as hosts for expression are well known in the art and include, but are not limited to, immortalized cell lines available from the American Type Culture Collection (ATCC), including but not limited to Chinese hamster ovary (CHO) cells, CHO-LARGE cells, HEK293 cells, HeLa cells, baby hamster kidney (BHK) cells, monkey kidney cells (COS), human hepatocellular carcinoma cells (e.g., Hep G2), and other cell lines standard in the art.

The rAAV genomes provided herein lack AAV rep and cap DNA. Recombinant AAV genomes provided herein comprise a polynucleotide encoding a therapeutic protein as described above and one or more AAV ITRs flanking the polynucleotide. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. To promote skeletal muscle specific expression, AAV1, AAV5, AAV6, AAV8 or AAV9 may be used.

DNA plasmids are provided that comprise rAAV genomes. The DNA plasmids are transferred to cells permissible for infection with a helper virus of AAV (including, but not limited to, adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV ITRs and rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, Current Opinions in Biotechnology, 1533-1539 (1992); and Muzyczka, Curr. Topics in Microbial. And Immunol., 158:97-129 (1992). Various approaches are described in Ratschin et al., Mol. Cell. Biol., 4:2072 (1984); Hermonat et al., Proc. Natl. Acad. Sci. USA, 81:6466 (1984); Tratschin et al., Mol. Cell. Biol., 5:3251 (1985); Mclaughlin et al., J. Virol., 62:1963 (1988); Lebkowski et al., Mol. Cell. Biol., 7:349 (1988); Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Human Gene Therapy, 4:609-615 (1993); Clark et al., Gene Therapy 3:1124-1132 (1996); U.S. Pat. Nos. 5,786,211; 5,871,982; 6,258,595; and McCarty, Mol. Ther., 16(10): 1648-1656 (2008). The foregoing documents are hereby incorporated by reference in their entirety herein, with particular emphasis on those sections of the documents relating to rAAV production.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV, which herein are replication-deficient, infectious, encapsidated viral particles (rAAV), comprise a rAAV genome. A rAAV encodes a therapeutic protein described herein. The rAAV genomes lack AAV rep and cap DNA, that is, there is no AAV rep or cap DNA between the ITRs of the rAAV genomes.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118; and WO 98/09657.

In another embodiment, the invention contemplates compositions comprising rAAV or therapeutic protein described herein. Compositions of the invention comprise rAAV or therapeutic protein in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods described herein will vary depending, for example, on the particular rAAV, the mode of administration, the treatment goal, the individual, and the cell type(s) being targeted, and may be determined by methods standard in the art. Titers of rAAV may range from about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg) as understood in the art.

Methods of transducing a target cell with rAAV, in vivo or in vitro, are contemplated by the invention. The in vivo methods comprise the step of administering an effective dose, or effective multiple doses, of a composition comprising a rAAV described herein to an animal (including a human patient) in need thereof.

Dosages and the frequency of administration of therapeutic proteins described herein may vary according to such factors as the route of administration, the particular therapeutic protein administered, and the size and general condition of the patient. Appropriate dosages can be determined by procedures known in the pertinent art, e.g., in clinical trials that may involve dose escalation studies. In view of these factors, a typical dose for a therapeutic protein described herein may range from about 0.1 pg/kg to up to about 30 mg/kg or more. Further, a dose may range from 0.1 pg/kg up to about 30 mg/kg, from 1 pg/kg up to about 30 mg/kg, from 10 pg/kg up to about 10 mg/kg, from about 0.1 mg/kg to 5 mg/kg, or from about 0.3 mg/kg to 3 mg/kg.

Methods of treating a patient with a therapeutic protein described herein are thus also provided. The methods comprise the step of administering an effective dose, or effective multiple doses, of a composition comprising a therapeutic protein described herein to an animal (including a human patient) in need thereof.

If the dose is administered prior to development of a disorder/disease, the administration is prophylactic. If the dose is administered after the development of a disorder/disease, the administration is therapeutic. An “effective dose” is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Methods described herein result in one or more of improved ambulation time, limb grip strength, decreased muscle pathology, and decreased neural pathology in a treated patient. Other endpoints achieved by methods described herein are one or more of increased muscle fiber size, decreased number of small oxidative fibers, correction of muscle atrophy, increased muscular force, and increased muscle regeneration in the treated patient. Dystroglycanopathies and laminin-deficient muscular dystrophies are contemplated for prevention or treatment according to methods of the invention.

Combination therapies are also contemplated by the invention. Combination therapies as used herein includes both simultaneous treatment, or sequential treatments. Combinations of methods described herein with standard medical treatments are specifically contemplated, as are combinations with novel therapies.

Administration of an effective dose of the compositions of rAAV or therapeutic protein may be by routes standard in the art including, but not limited to, intramuscular, intraparenteral, intravenous, intrathecal, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the therapeutic proteins.

In particular, actual administration of rAAV of the present invention may be accomplished by using any physical method that will transport the rAAV recombinant vector into the target tissue of an animal. Administration according to the invention includes, but is not limited to, injection into muscle, the bloodstream and/or directly into the liver. Simply resuspending a rAAV in phosphate buffered saline or lactated Ringer's solution has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

For purposes of intramuscular injection, solutions of rAAV or therapeutic protein in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms of rAAV or therapeutic protein suitable for systemic (e.g., intravenous) injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained expression of a therapeutic protein described herein. The present invention thus provides methods of administering rAAV which express a therapeutic protein described herein to a patient, preferably a human being. These methods include transducing tissues (including, but not limited to, tissues such as muscle, organs such as liver and brain, and glands such as salivary glands) with one or more rAAV of the present invention.

Muscle tissue is an attractive target for in vivo DNA delivery, because it is not a vital organ and is easy to access. The invention contemplates sustained expression of therapeutic protein described herein from transduced muscle cells.

By “muscle cell” or “muscle tissue” is meant a cell or group of cells derived from muscle of any kind [for example, skeletal muscle and smooth muscle (e.g., from the digestive tract, urinary bladder, blood vessels or cardiac tissue)]. Such muscle cells may be differentiated or undifferentiated, such as myoblasts, myocytes, myotubes, cardiomyocytes and cardiomyoblasts.

The term “transduction” is used to refer to the administration/delivery of therapeutic protein to a recipient cell either in vivo or in vitro, via a rAAV of the invention resulting in expression of therapeutic protein by the recipient cell.

Thus, methods are provided herein of administering an effective dose (or doses administered essentially simultaneously or doses given at intervals) of rAAV that encode a therapeutic protein described herein to a patient in need thereof.

Methods are also provided herein of administering an effective dose (or doses administered essentially simultaneously or doses given at intervals) of a therapeutic protein described herein to a patient in need thereof.

### EXAMPLES

Aspects and embodiments of the invention are illustrated by the following examples. Example 1 describes constructs encoding therapeutic proteins of the disclosure. Example 2 describes recombinant expression of the therapeutic proteins in cultured host cells. Example 3 describes experiments demonstrating heparin-binding domain targets LAMA2(g1-G50 to the muscle of wild type mice. Example 4 describes experiments to demonstrate efficacy of AAV-mediated HBEGF-LAMA2(G1-5) expression in reducing symptoms and pathology in the dy^{W}/dy^{W} mouse model of MDC1A. Example 5 describes experiments to demonstrate efficacy of AAV-mediated HBEGF-DAG1(α) expression in reducing symptoms and pathology in the mouse models of dystroglycanopathy. Example 6 describes properties of (domains of) sHBEGF contemplated herein as useful for its application as a linker domain in therapeutic proteins described herein and as a trophic factor in various methods described herein.

### Example 1

### Constructs Encoding Therapeutic Proteins

Six exemplary DNA constructs encoding therapeutic proteins including an HBEGF EGF domain were generated as follows:

HB-EGF (ending at heparin binding domain)-LAMA2 G1-G5 (encoded by the polynucleotide of FIG. 3),
HB-EGF (complete soluble form)-LAMA2 G1-G5 (encoded by the polynucleotide of FIG. 4),
HB-EGF (ending at heparin binding domain)-LAMA2 G3-G5 (encoded by the polynucleotide of FIG. 5),
HB-EGF (complete soluble form)-LAMA2 G3-G5 (encoded by the polynucleotide of FIG. 6),
HB-EGF (ending at heparin binding domain)-DAG1 (native processed alpha DG gene) (encoded by the polynucleotide of FIG. 7), and
HB-EGF (complete soluble form)-DAG1 (native processed alpha DG gene) (encoded by the polynucleotide of FIG. 8).

The constructs were expressed from plasmids in CHO cells. CHO cells were transfected with plasmids containing one of the constructs or mock-transfected (-).

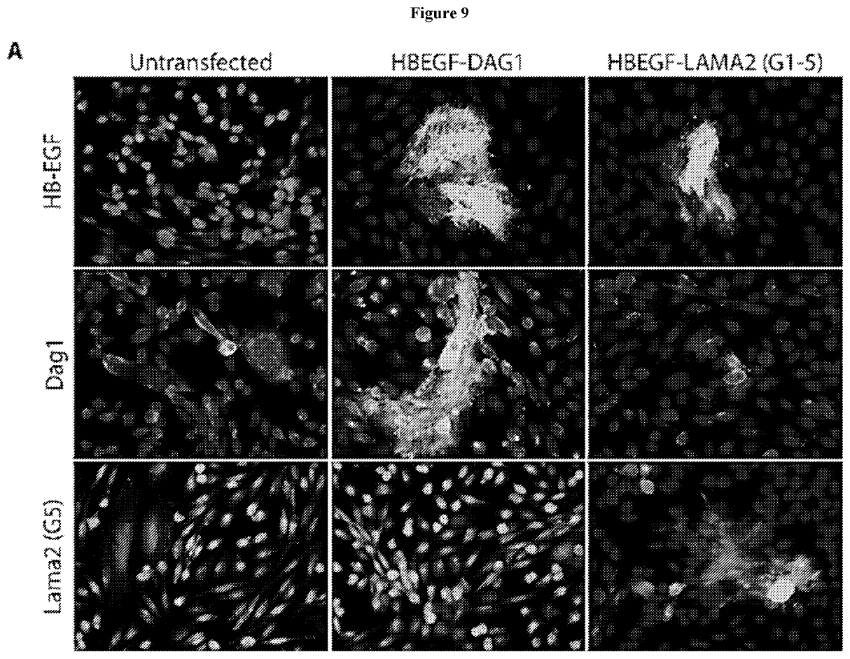

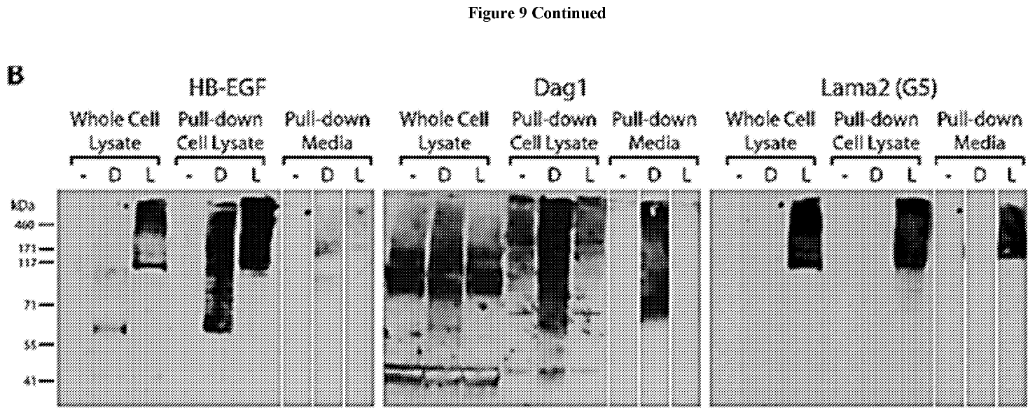

The transfected CHO cells were stained with antibodies against HB-EGF, dystroglycan, or laminin-α2 G5 domain. Results are shown in FIG. 9A. Also, culture media was collected from each plate 48 hours post-transfection and cell lysis was performed. Heparin-agarose pull-down was performed on both cell lysate and culture media and loaded in Western blot along with whole cell lysate. Results are shown in FIG. 9B.

### Example 2

### Recombinant Expression of Therapeutic Proteins in Cultured Host Cells

The constructs of Example 1 were also subcloned into a plasmid to produce AAV9 vectors encoding the therapeutic proteins.

AAV vectors carrying one of the therapeutic genes of Example 1 under the transcriptional control of the cytomegalovirus (CMV) promoter were produced.

rAAV vectors were produced by a modified cross-packaging approach whereby the AAV type 2 vector genome can be packaged into multiple AAV capsid serotypes [Rabinowitz et al., J Virol. 76 (2):791-801 (2002)]. Production was accomplished using a standard three plasmid DNA/CaPO4 precipitation method using HEK293 cells. HEK293 cells were maintained in DMEM supplemented with 10% fetal bovine serum (FBS) and penicillin and streptomycin. The production plasmids were: (i) plasmids encoding the therapeutic proteins, (ii) rep2-capX modified AAV helper plasmids encoding cap serotype 9 isolate, and (iii) an adenovirus type 5 helper plasmid (pAdhelper) expressing adenovirus E2A, E4 ORF6, and VA I/II RNA genes. A quantitative PCR-based titration method was used to determine an encapsidated vector genome (vg) titer utilizing a Prism 7500 Taqman detector system (PE Applied Biosystems). [Clark et al., Hum Gene Ther. 10 (6): 1031-1039 (1999)]. A final titer (vg ml⁻¹) was determined by quantitative reverse transcriptase PCR using the specific primers and probes utilizing a Prism 7500 Real-time detector system (PE Applied Biosystems, Grand Island, NY, USA). Aliquoted viruses were kept at −80° C. until use.

The rAAV set out in Table 2 were used in the experiments described herein.

TABLE 2

| | Membrane | | | | |
|---|---|---|---|---|---|
| | linker | | Denoted | SEQ | rAAV |
| AAV | (first | Second | herein | ID | genome |
| construct | domain) | Domain | as | NO: | nt's |
| rAAV.CMV.HB. | Heparin | Laminin | HB-LAMA2 | 2 | Nt. |
| LAMA2(G1-G5) | binding | alpha 2 | (G1-G5) | | 3590-8215 |
| | domain | G1-G5 | | | |
| | only | | | | |
| rAAV9.CMV. | Complete | Laminin | HBEGF- | 4 | Nt. |
| HBEGF.LAMA2 | soluble | alpha 2 | LAMA2 | | 3590-8341 |
| (G1-G5) | form | G1-G5 | (G1-G5) | | |
| rAAV.CMV.HB. | Heparin | Laminin | HB-LAMA2 | 6 | Nt. |
| LAMA2(G3-G5) | binding | alpha 2 | (G3-G5) | | 3609-6929 |
| | domain | G3-G5 | | | |
| | only | | | | |
| rAAV.CMV. | Complete | Laminin | HBEGF- | 8 | Nt. |
| HBEGF.LAMA2 | soluble | alpha 2 | LAMA2 | | 3590-7036 |
| (G3-G5) | form | G3-G5 | (G3-G5) | | |
| rAAV.CMV.HB. | Heparin | DAG1 | HB-DAG1 | 10 | Nt. |
| DAG1 | binding | | | | 3590 to |
| | domain | | | | 6340 |
| | only | | | | |
| rAAV.CMV. | Complete | DAG1 | HBEGF. | 12 | Nt. |
| HBEGF.DAG1 | soluble | | DAG1 | | 3590 to |
| | form | | | | 6049 |

### Example 3

### Heparin-Binding Domain Targets LAMA2(G1-G5) to the Muscle in Wild Type Mice

Wild type mice were injected intramuscularly in the gastrocnemius muscle with 5×10¹¹ vg pf rAAV9.CMV vectors containing HBEGF, HBEGF.LAMA2(G1-G5), HBEGF.LAMA2(G3-G5), HB.LAMA2(G1-G5), HB.LAMA2(G3-G5), or LAMA2(G1-G5) (see Table 2). Cells were stained with antibody specific to human HB-EGF or recombinant G5 domain of LAMA2. Mock injected mice (buffer alone) are shown as a negative control. 4H8-2 is an anti-laminin antibody to show muscle cells in the sections.

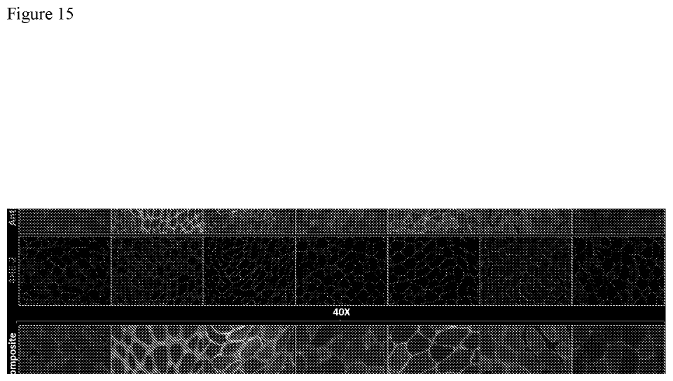

As shown in FIG. 15, HBEGF and HBEGF.LAMA2(G1-G5) reduced muscle growth and/or induced mild muscle atrophy, much as we had previously shown for overexpression of HBEGF, while IM injection of rAAV9.CMV.HB.LAMA2(G1-G5) lead to secretion and localization of LAMA2(G1-G5) protein in the extracellular matrix. In addition, HB. LAMA2(G1-G5)-expressing muscles appeared larger than normal wild type muscles. HB.LAMA2(G3-G5) showed lower overall protein staining than HB.LAMA2(G1-G5). The ECM targeting function of the HB domain of HBEGF allows for secretion and targeting of LAMA2(G1-G5) protein to the muscle extracellular matrix. By contrast, expression of LAMA2(G1-G5) without the HB domain led to very poor protein production and no detectable ECM localization. Thus, the constructs comprising the HB domain alone, rather than the full HB-EGF domain serves the purpose of targeting LAMA2(G1-G5) to the muscle ECM, and successfully does so without having the negative consequences of EGF signaling, as this construct has the EGF domain from HBEGF deleted.

The pre-pro peptide portion of HBEGF, which is still present in the HB construct, may also improve protein folding and/or expression for LAMA2(G1-G5) relative to LAMA2(G1-G5) alone, which only contains the signal peptide from HBEGF. Last, HB.LAMA2(G1-G5), when localized appropriately, may improve muscle growth, even in normal muscles.

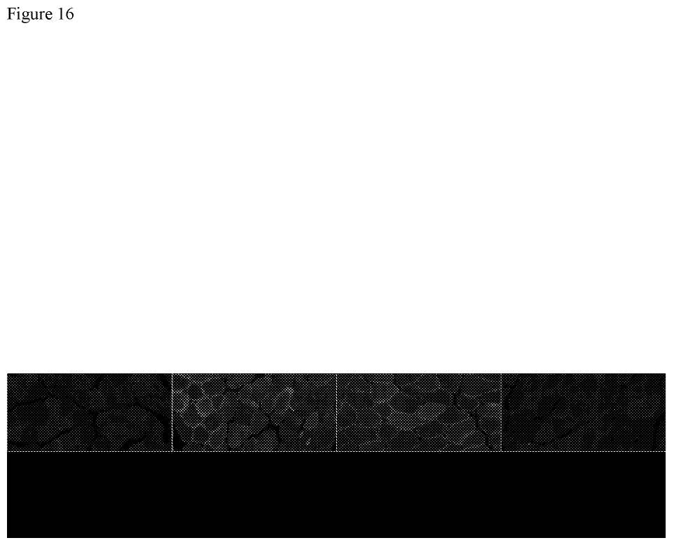

FIG. 16 shows staining of HBEGF.LAMA2(G1-G5), HB.LAMA2(G1-G5) and LAMA2(G1-G5) using an antibody to human HBEGF protein and an antibody to the G5 domain of human LAMA2. This data confirmed that expression of LAMA2(G1-G5) alone leads to very poor protein production in muscle, while inclusion of the HB domain improves expression for LAMA2(G1-G5), which was visualized with an anti-laminin antibody as well as an HBEGF antibody.

### Example 4

### Efficacy of AAV9-Mediated HBEGF-LAMA2(G1-5) Expression in Reducing Symptoms and Pathology in the dy^{W}/dy^{W} Mouse Model of MDC1A

dy^{W}/dy^{W} mice [Nonaka, Lab Anim Sci., 48(1): 8-17 (1998)] have a loss-of-function mutation in Lama2, resulting in impaired laminin-α2 production, similar to MDC1A pathogenesis. Dy^{W}/dy^{W} mice have decreased size, grip strength, and lifespan compared to wild-type mice. They display muscle atrophy, dystrophic muscle pathology, and severely impaired ambulation by three months of age. As such, these mice are an appropriate and robust model for testing MDC1A therapy.

To demonstrate the therapeutic efficacy of the LAMA2 expressing rAAV genomes provided in Example 2, 8 dy^{W}/dy^{W} pups were injected intravenously through the facial vein at postnatal day 1 with either a low dose, 10¹¹ viral genomes (vg), or a high dose, 10¹² vg, rAAV9.CMV.HBEGF.LAMA2(G1-G5), or rAAV9.CMV.HB.LAMA2(G1-G5) or rAAV.CMV.HB.LAMA2(G3-G5). Mock injections of AAV buffer in control dy^{W}/dy^{W} pups were also performed.

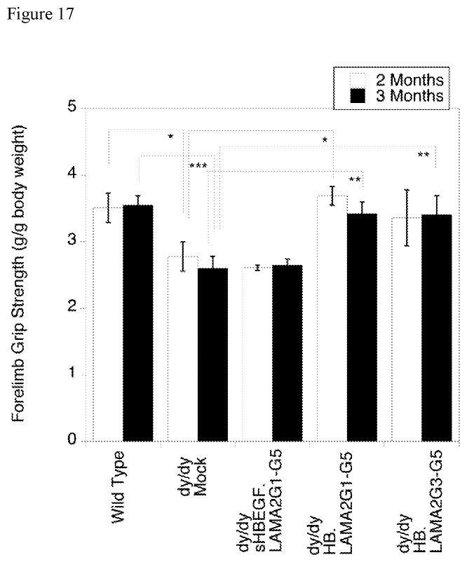

At 2 and at 3 months post-injection, grip strength in the forelimb muscles was analyzed (FIG. 17). At 4 months of age, the mice were euthanized and limb muscles were dissected and analyzed for expression of recombinant protein. As shown in FIG. 17, both HB.LAMA2(G3-G5) and HB.LAMA2(G1-G5) prevented loss of grip strength in dy/dy mice, showing a significant change from mock-treated dy/dy mice and bringing strength values to within the range seen in untreated wild type mice of the same age. Thus, both HB-LAMA2(G3-G5) and HB-LAMA2(G1-G5) show a therapeutic effect in the dy/dy model for MDC1A. In this experiment, HB.LAMA2(G3-G5) did not reach significance at 2 months relative to mock-treated dy/dy mice, while HB.LAMA2(G1-G5) did.

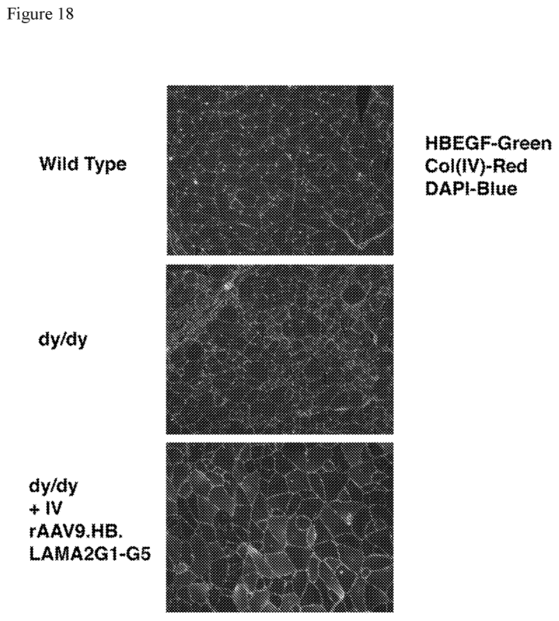

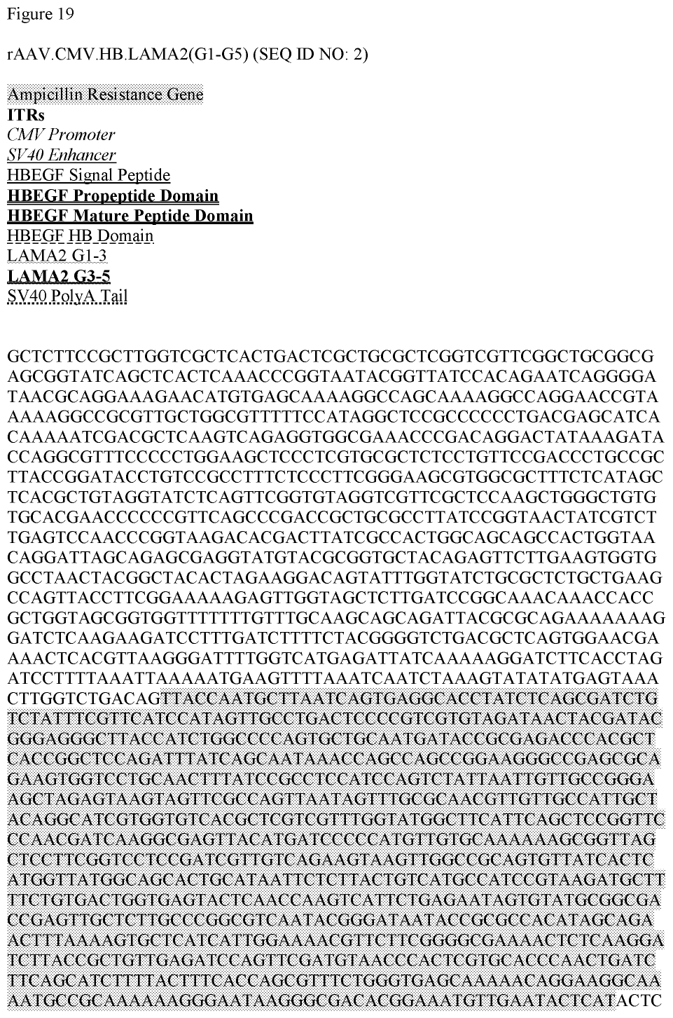

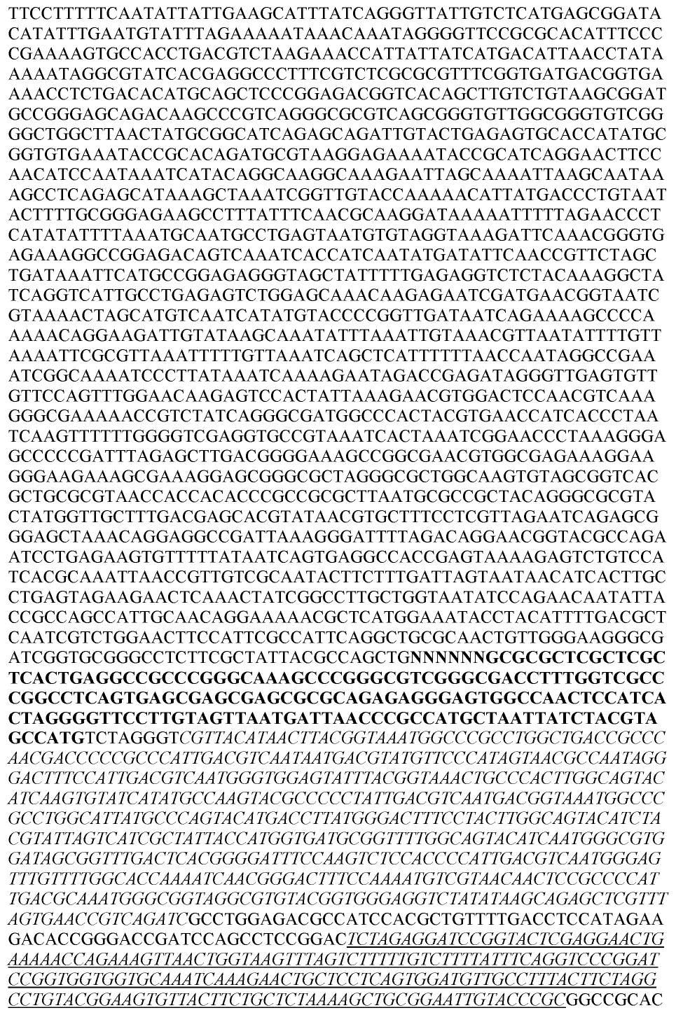

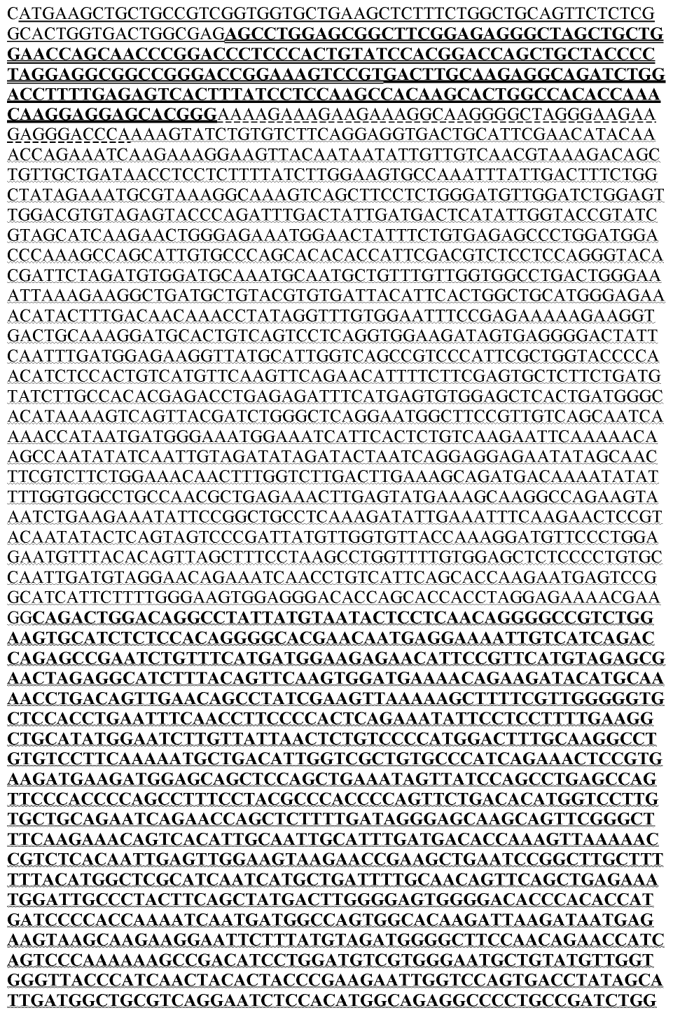

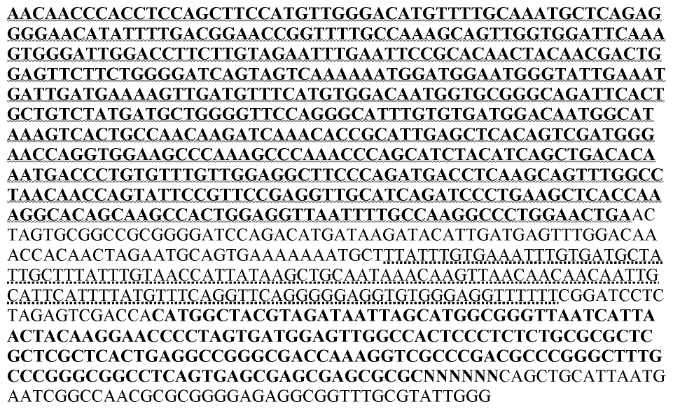

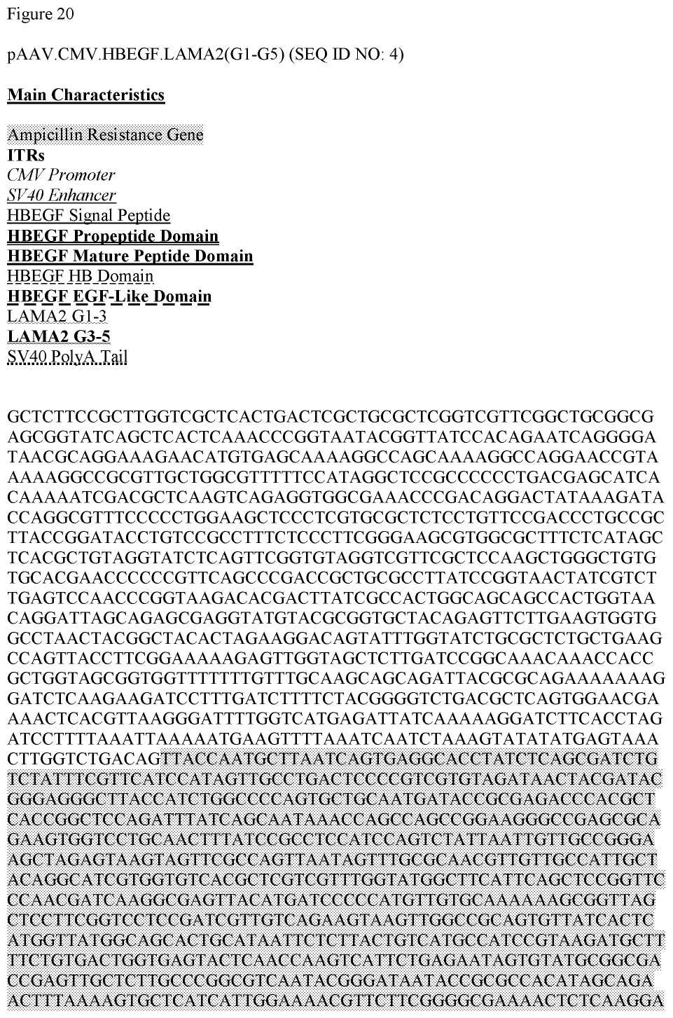

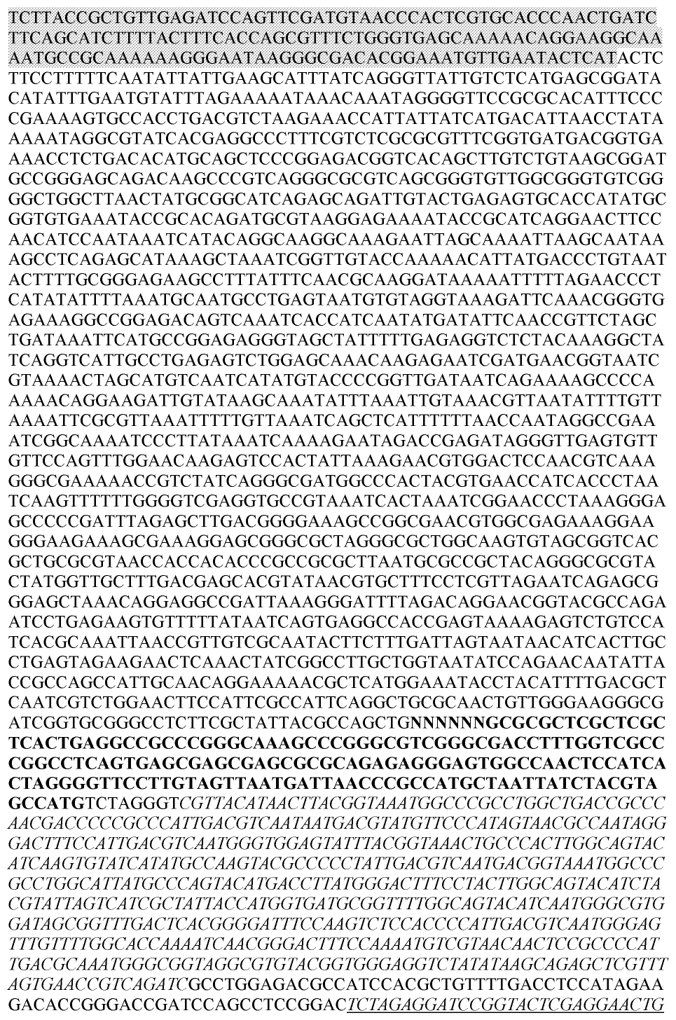

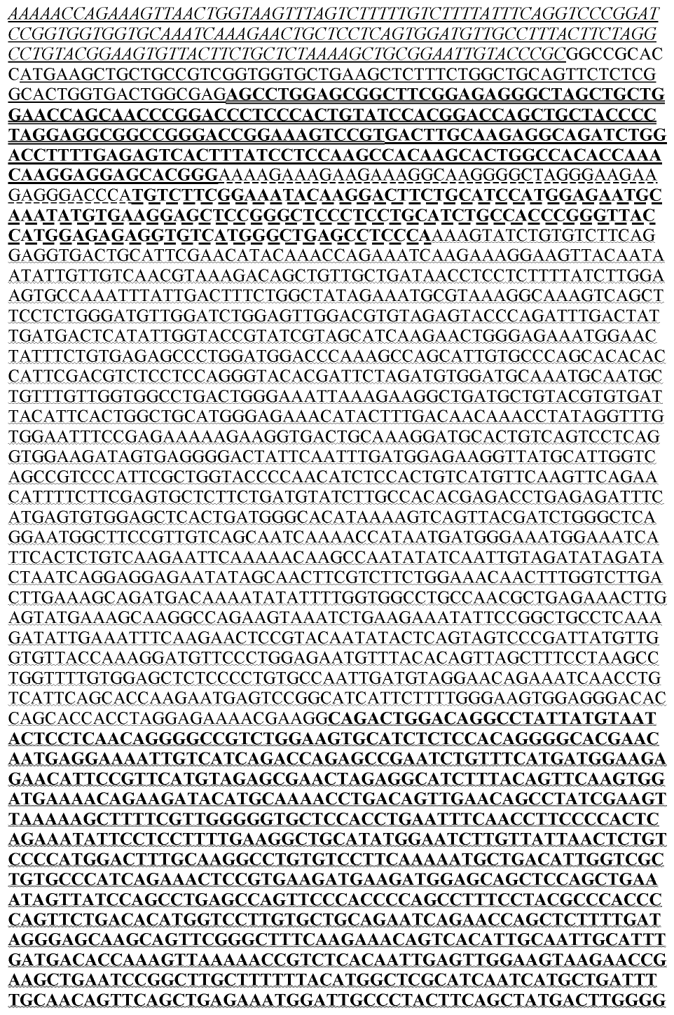

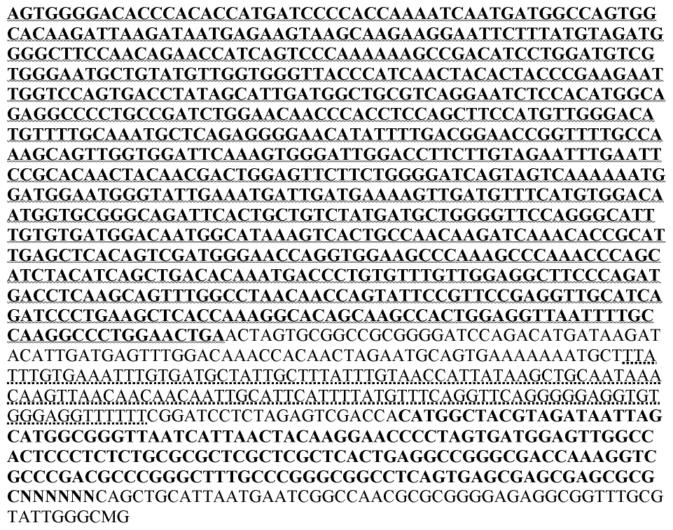

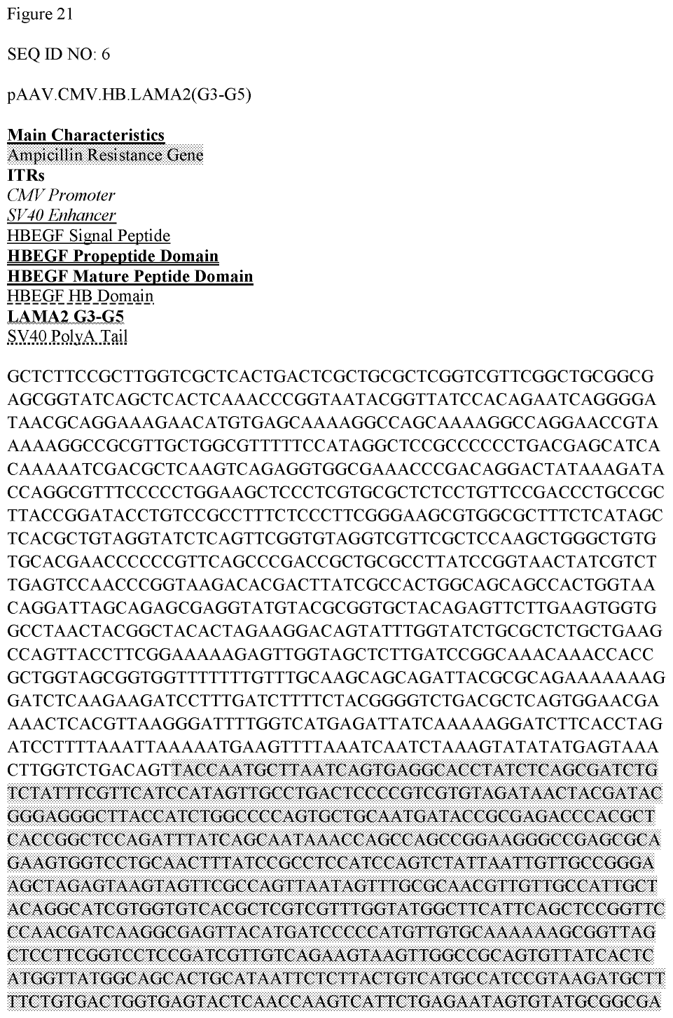

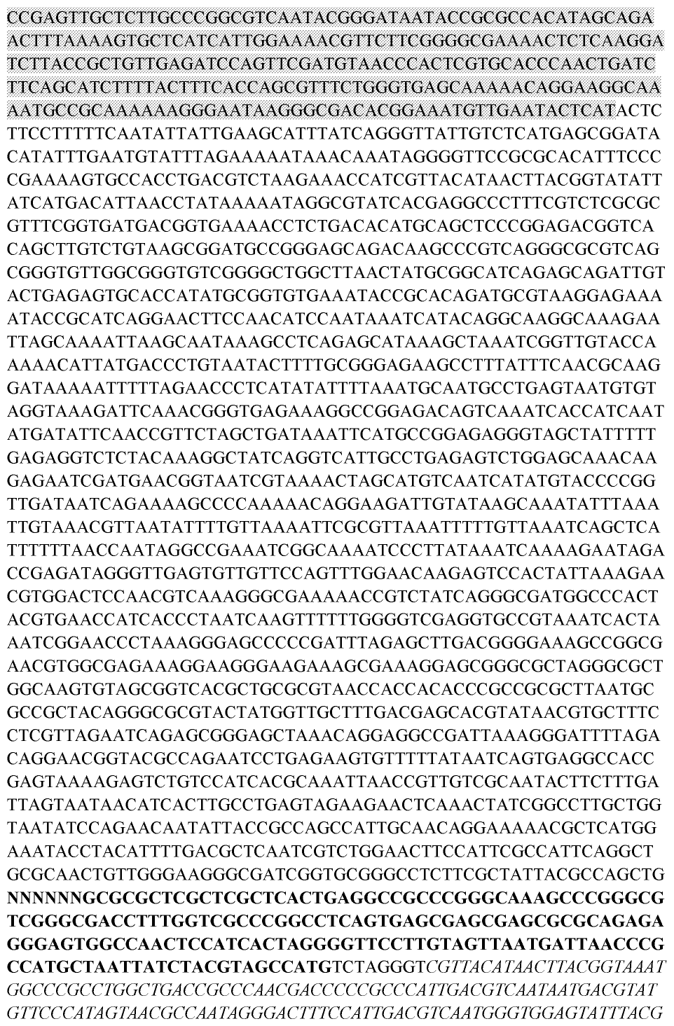

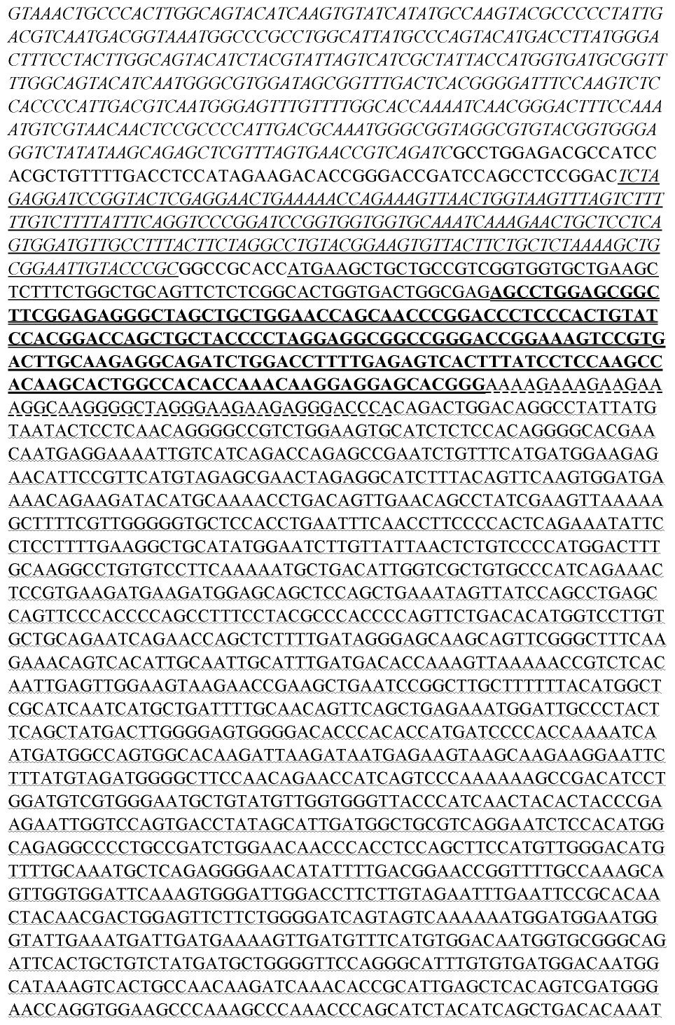

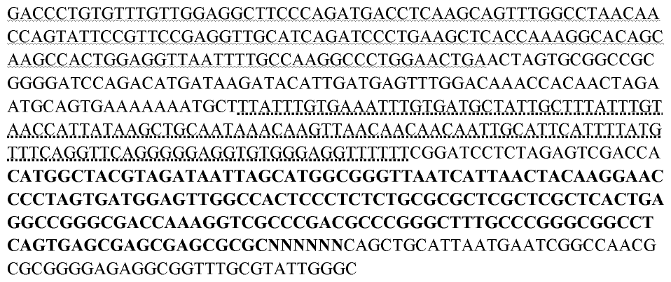

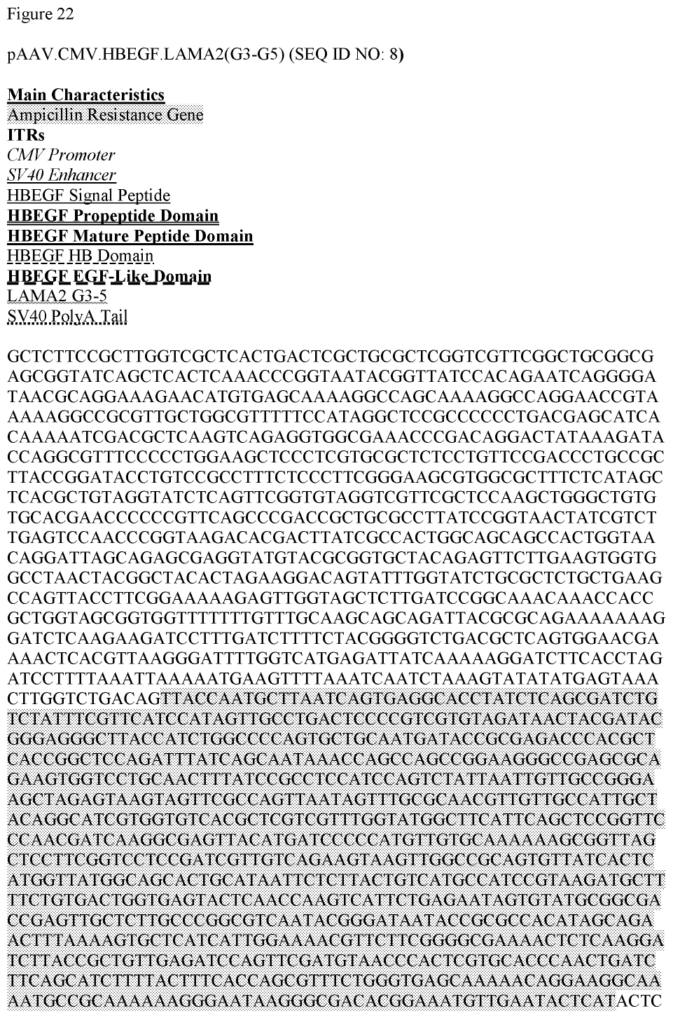

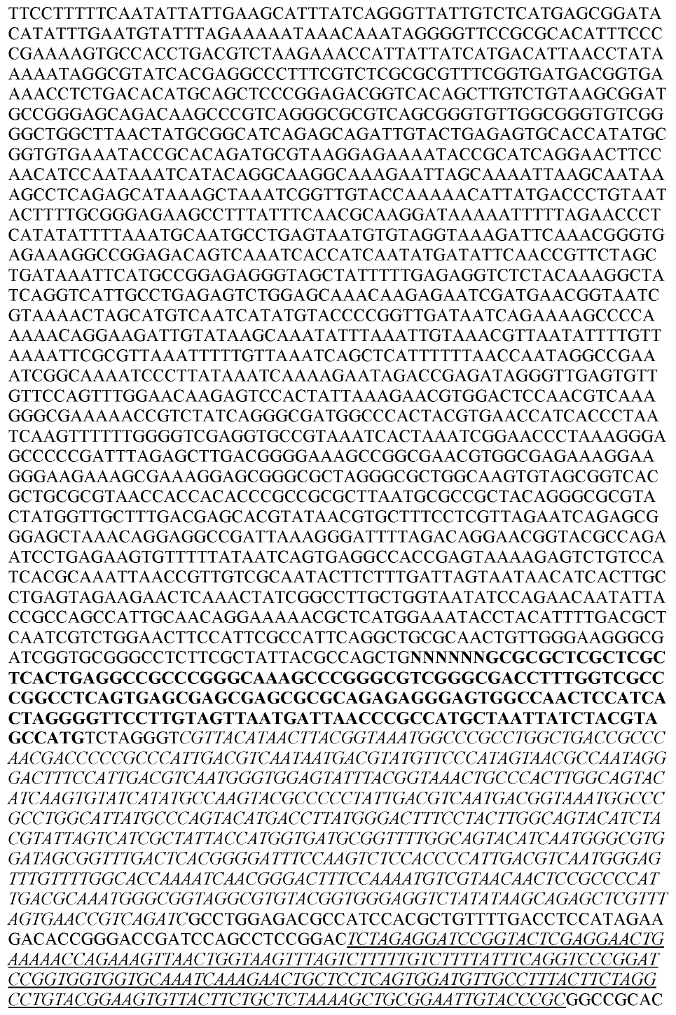

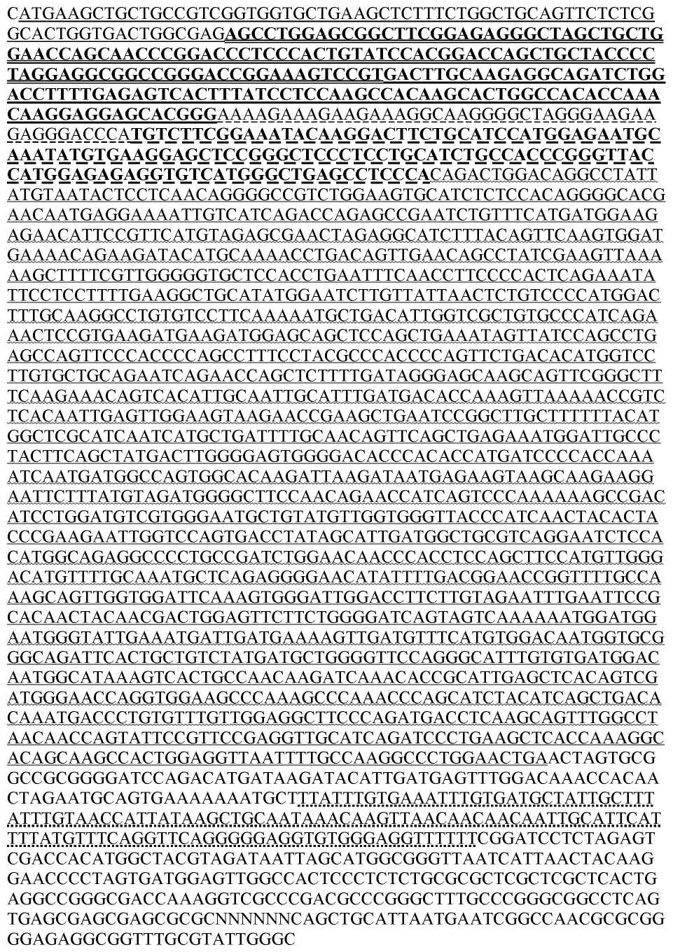

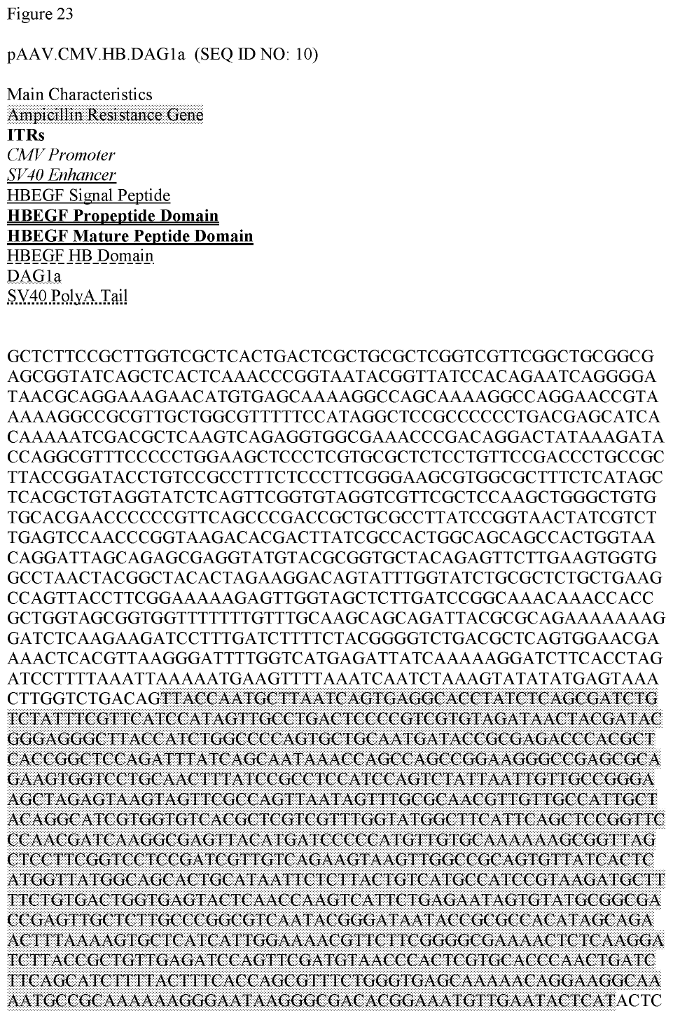

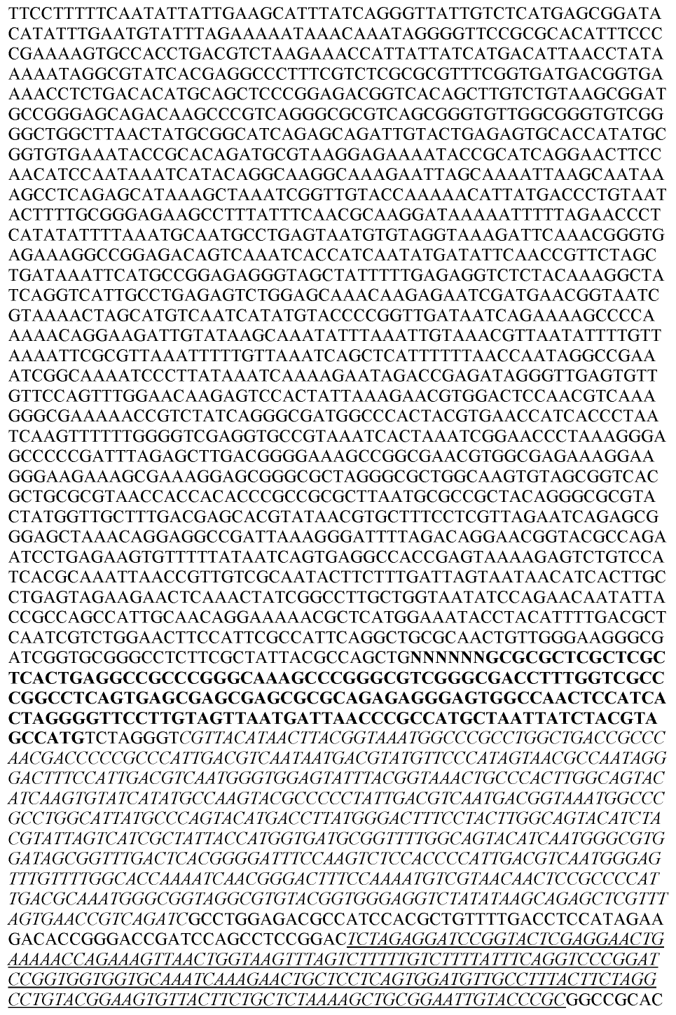

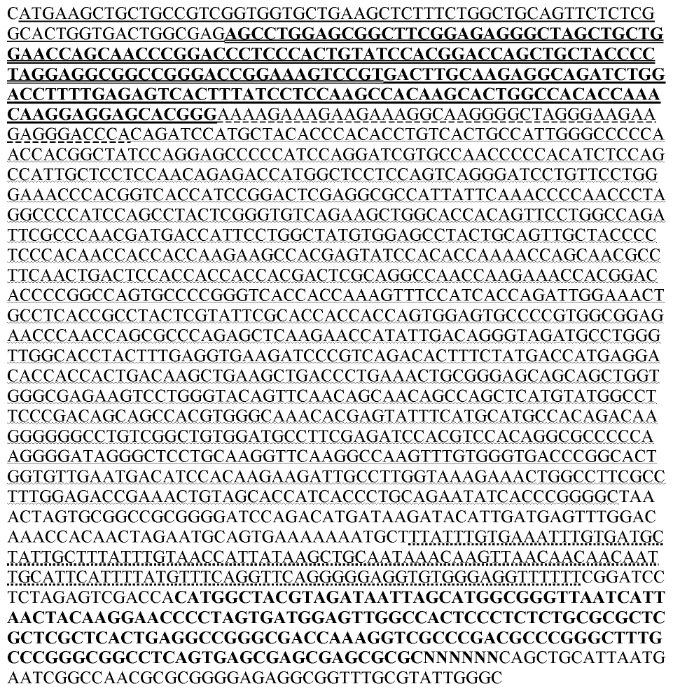

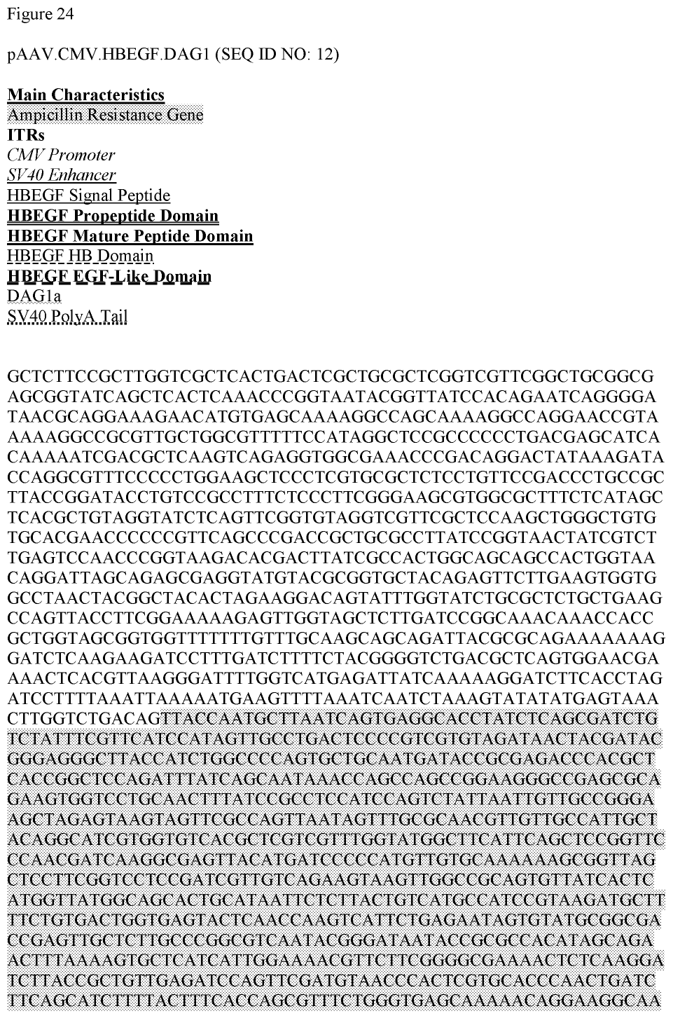

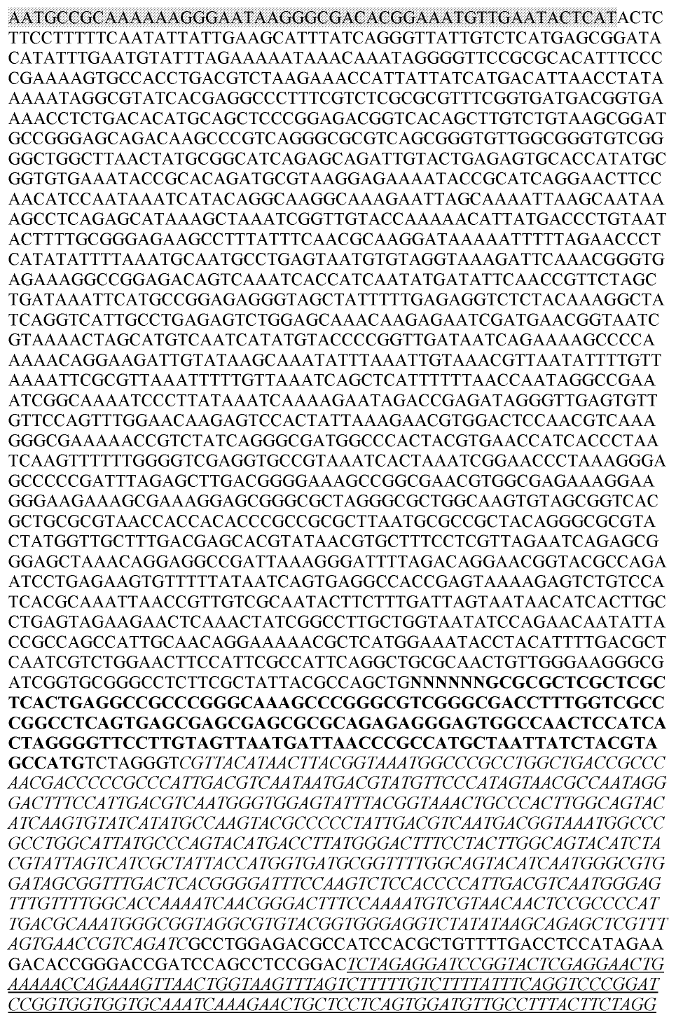

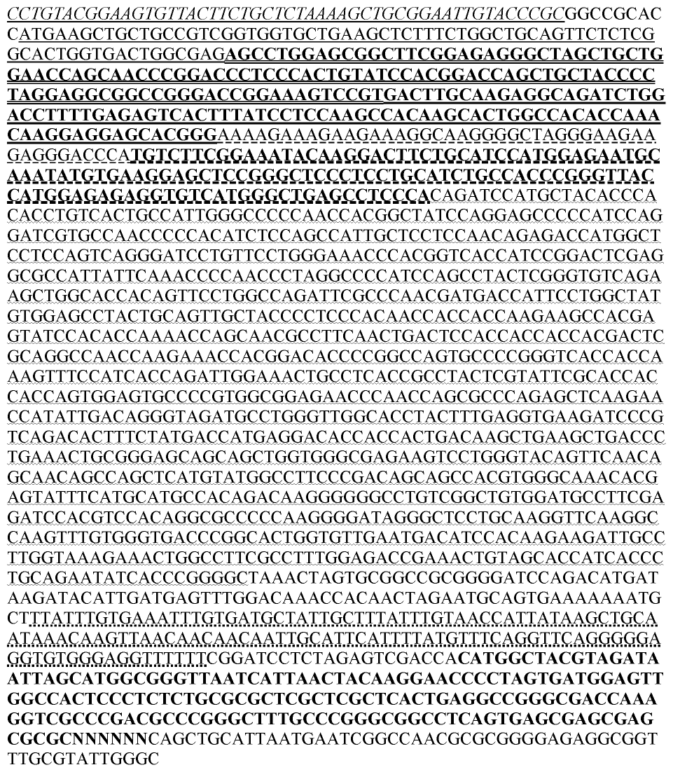

The role of transgene expression in preventing muscle pathology by comparing the percentage of myofibers with central nuclei, myofiber diameter and area, and variance in myofiber diameter in treated dy/dy mice was also analyzed. Muscle pathology intransgene-expressing myofibers was compared to the same pathology measures in non-expressing myofibers using the triceps muscle. This experiment demonstrated that expression of HB.LAMA2(G1-G5) increased muscle size. An example of staining showing such changes is shown in FIG. 18. When quantified across all muscles, the average cross-sectional muscle area was 2328 mm² in expressing myofibers versus 1082 mm² in nonexpressing myofibers (n=4 muscles each with 400 myofibers analyzed per muscle), which was a two-fold average increase in muscle size with treatment. The variance in myofiber diameter index (Diameter SD/Mean X1000) was reduced from 620 in non-expressing muscles to 431 in expressing muscles (250 or lower is considered normal), and the percentage of myofibers with central nuclei, an indicator of a cycle of muscle degeneration and regeneration, was reduced from 28% in non-expressing myofibers to 14% in expressing ones (n=2 each).

While not reduced to zero pathology, it is important to remember that AAV requires 3-4 weeks to achieve maximal gene expression, so some pathology will develop in these animals prior to when therapeutic transgene expression occurs. In all such experiments, the average level of muscle transduction was 26±1% (n=4 triceps muscles analyzed, 400 fibers each). The take home from these pathological measures is that HB-LAMA2(G1-G5) not only appears to prevent, at least in part, muscle damage in dy/dy muscles, but it also increases muscle growth back to, and perhaps beyond, wild type levels.

### Example 5

### Efficacy of AAV-Mediated HBEGF-DAG1(α) Expression in Reducing Symptoms and Pathology in Mouse Models of Dystroglycanopathy

Mice lacking dystroglycan or the α-dystroglycan-glycosylating enzyme, fukutin, encoded by the Fktn gene, are embryonic lethal, and cannot be used to study dystroglycanopathy therapy. Myf5Cre-Fktn^{loxP} mice [Kanagawa et al., Hum Mol Genet., 22(15):3003-3015 (2013)], in which Fktn deletion is restricted to skeletal muscle are used to demonstrate efficacy. Myf5Cre-Fktn^{loxP} mice have decreased body weight, grip strength, and lifespan compared to wild-type mice. They also display dystrophic muscle pathology.

To demonstrate the therapeutic efficacy of rAAV9.CMV.HBEGF-DAG1(α) in the Myf5Cre-Fktn^{loxP} mouse model, the same injection protocol and assessments are performed as described in Example 3.

Another mouse model of dystroglycanopathy the Large-vls mouse mutant (Lee et al., Mol. Cell. Neurosci. 30: 160-172, 2005). Several Large vls mice were IM injected with 1×10¹² vg rAAV9.CMV.HB-DAGI IV via the facial vein at P1. The tests of grip strength of these mice suggest potential improvement. Of the 4-7 animals analyzed per group at 2 months, forelimb grip strength is reduced from 4.7±0.2 g/g in wild type to 3.8±0.1 g/g in untreated Large vls mice (p=0.0005), while pAAV9.CMV.HB-DAG1(α) treatment of Large vls mice (IV with 1×10¹² vg at P1) showed improvement in grip strength, to 4.4±0.3 g/g (p=0.06 versus mock-treated Large vls). This data is very close to significance, and a significant difference may be achieved with additional measures.

### Example 6

### SHBEGF as a Linker Protein and Trophic Factor

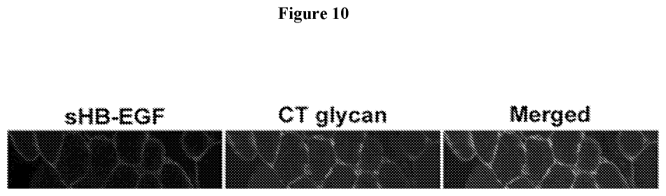

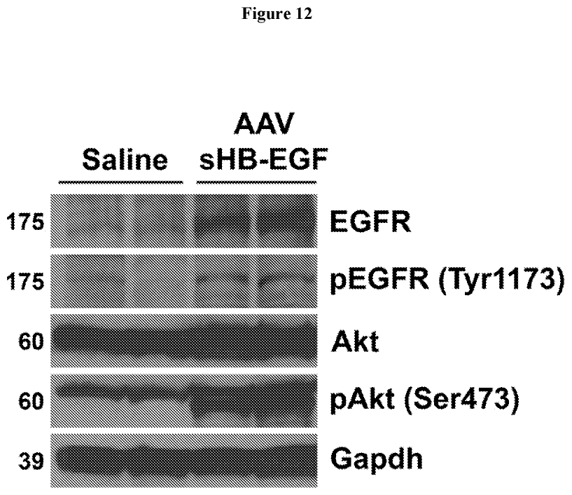

Using the heparin-binding domain and EGF-like domain of soluble HBEGF (SHBEGF) in various exemplary therapeutic proteins described herein provides a dual benefit to patients. Including both domains adds increased muscle membrane stability from the inclusion of the heparin-binding domain along with LAMA2(G1-5) or DAG1(α), and then inclusion of the EGF-like domain additionally provides a stimulus for muscle regeneration. FIGS. 10, 11 and 12 show sHBEGF activates an Akt kinase pathway in muscle and increases the expression of muscle regeneration markers. Expression of activated Akt kinase in muscle has previously been shown to stimulate profound muscle growth, akin to what is seen with myostatin inhibitors. The presence of the EGF-like domain of HBEGF in various therapeutic proteins described herein, therefore, adds an additional therapeutic effect for treatment of the diseases described herein.

The gastrocnemius muscle on the left side of 5-week-old male C57BL/6J mice was injected with 5×10¹⁰ vector genomes of r(ds)AAV9.CMV.HB-EGF or r(ds)AAV9.CMV.sHB-EGF in a volume of 50 μL sterile PBS using a 0.3 mL insulin syringe near the midpoint of the muscle. Muscles on the contralateral (right) side of the mouse were mock-injected with an identical volume of sterile PBS. At 4- or 12-weeks post-injection, mice were sacrificed and dissected. Gastrocnemius muscles were embedded in O.C.T. Compound (Fisher Scientific, Pittsburgh, PA) and snap-frozen in liquid nitrogen-cooled isopentane.

sHB-EGF expression was visualized using an antibody that recognizes sHB-EGF and co-stained with either an antibody to Galgt2 protein or the CT glycan. sHB-EGF was expressed along the sarcolemmal membrane of skeletal myofibers in muscles analyzed at 4 weeks post-injection with r(ds)AAV9.CMV.sHB-EGF. FIG. 9 shows that sHB-EGF can be secreted from muscles and stick to the extracellular matrix, supporting its use as a linker protein.

FIG. 10 shows that sHB-EGF induces expression of therapeutic surrogate muscular dystrophy genes.

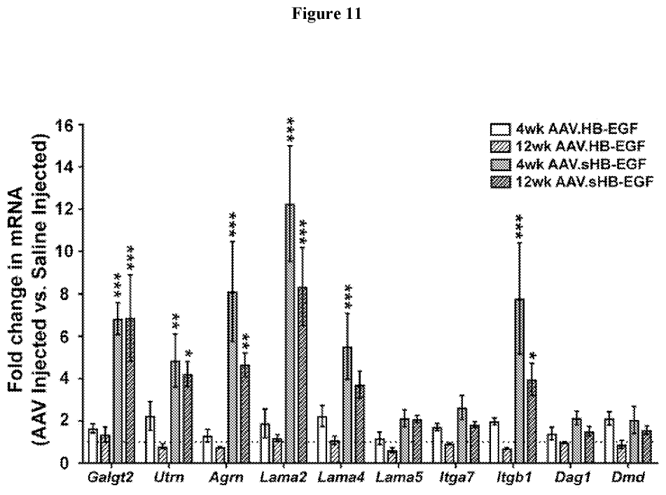

FIG. 11 shows that sHB-EGF induces Akt tyrosine kinase cascade in skeletal muscle and can stimulate muscle growth and regeneration.

While the present invention has been described in terms of specific embodiments, it is understood that variations and modifications will occur to those skilled in the art. Accordingly, only such limitations as appear in the claims should be placed on the invention.

All documents referred to in this application are hereby incorporated by reference in their entirety.

1. A polynucleotide encoding a protein comprising:
a) a first domain comprising the heparin-binding domain of Heparin-Binding Epidermal Growth Factor-Like Growth Factor (HBEGF), and a second domain comprising the G1-G5 domain of the human laminin alpha 2 (LAMA2) gene;
b) a first domain comprising the heparin-binding domain of HBEGF and the EGF-like domain of HBEGF, and a second domain comprising the G1-G5 domain of the human LAMA2 gene;
c) a first domain comprising the heparin-binding domain of HBEGF, and a second domain comprising the G3-G5 domain of the human LAMA2 gene; or
d) a first domain comprising the heparin-binding domain of HBEGF and the EGF-like domain of HBEGF, and a second domain comprising the G3-G5 domain of the human LAMA2 gene.

2. The polynucleotide of claim 1, wherein the first domain of the protein is encoded by the nucleotide sequence of SEQ ID NO: 13 or SEQ ID NO: 14 or wherein the second domain of the protein is encoded by the nucleotide sequence of SEQ ID NO: 15 or SEQ ID NO: 16.

3. (canceled)

4. The polynucleotide of claim 1, wherein the polynucleotide comprises
a) the nucleotide sequence of SEQ ID NO: 13 and SEQ ID NO: 15,
b) the nucleotide sequence of SEQ ID NO: 13 and SEQ ID NO: 16.

5. (canceled)

6. The polynucleotide of claim 1, wherein the polynucleotide comprises
a) the nucleotide sequence of SEQ ID NO: 14 and SEQ ID NO: 15 or
b) the nucleotide sequence of SEQ ID NO: 14 and SEQ ID NO: 16.

7. (canceled)

8. The polynucleotide of claim 1 wherein
(a) comprises i) nucleotides 14 to 3235 set forth in FIG. 3, ii) the nucleotide sequence of SEQ ID NO: 1, or iii) a nucleotide sequence encoding the amino acid sequence of SEQ ID NO: 19,
(b) comprises i) nucleotides 14 to 3361 set forth in FIG. 4, ii) the nucleotide sequence of SEQ ID NO: 3 or iii) a nucleotide sequence encoding the amino acid sequence of SEQ ID NO: 20
(c) comprising i) nucleotides 14 to 1930 set forth in FIG. 5, ii) the nucleotide sequence of SEQ ID NO: 5 or iii) a nucleotide sequence encoding the amino acid sequence of SEQ ID NO: 21
(d) comprising i) nucleotides 14 to 2056 set forth in FIG. 6, the nucleotide sequence of SEQ ID NO: 7 or iii) a nucleotide sequence encoding the amino acid sequence of SEQ ID NO: 22

9-12. (canceled)

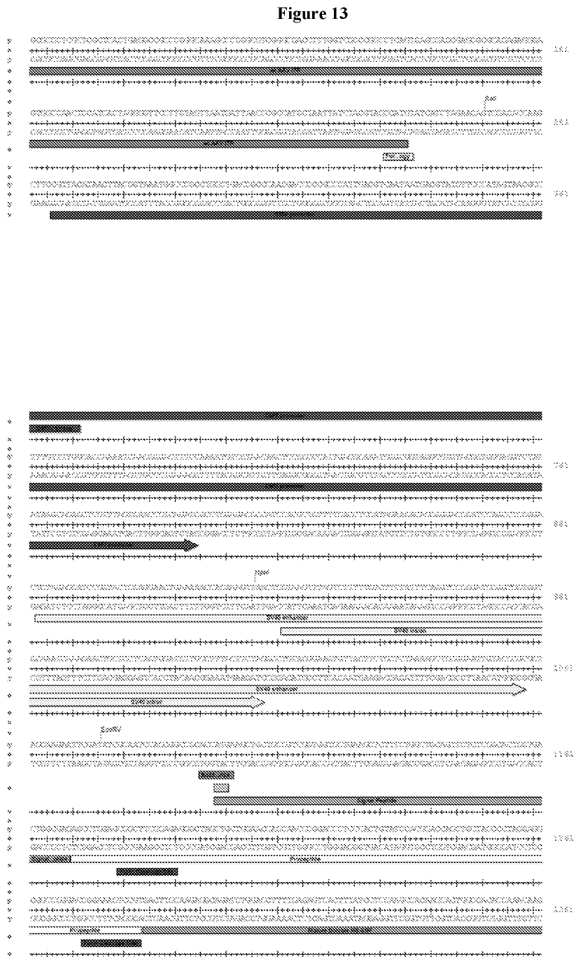

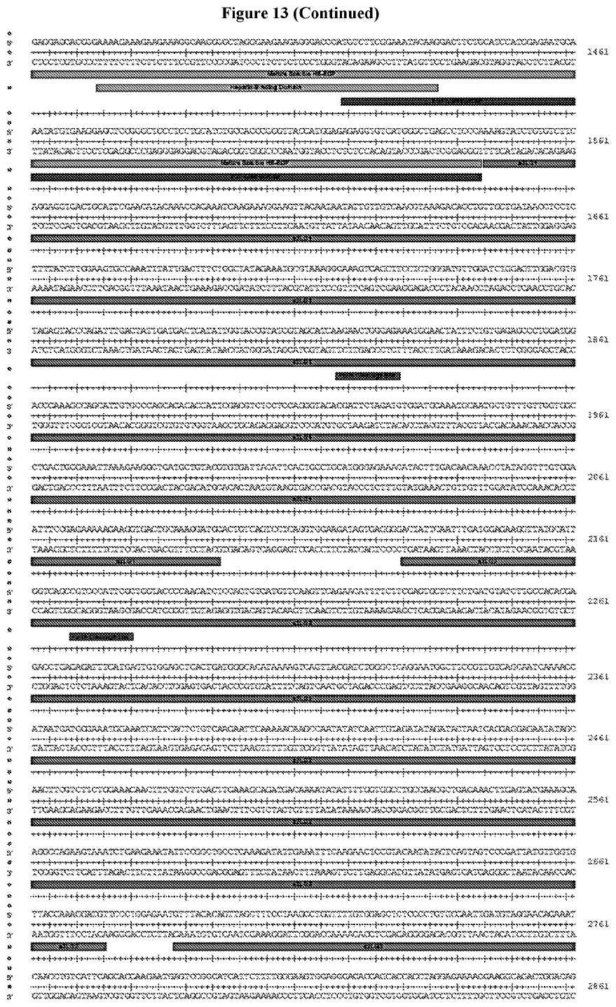

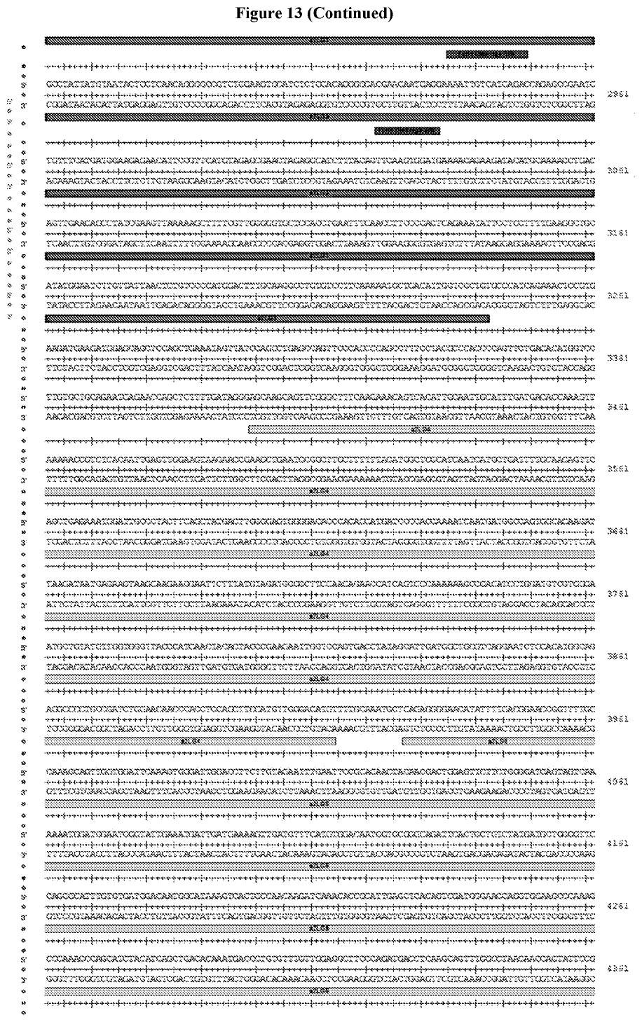

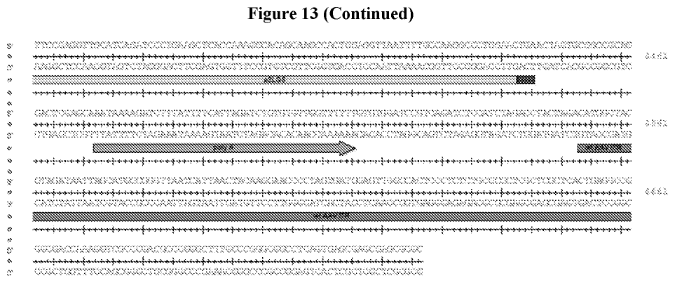

13. A recombinant adeno-associate virus (rAAV), wherein the genome of the rAAV comprises
(a) the polynucleotide sequence of SEQ ID NO: 1,
(b) the polynucleotide sequence of SEQ ID NO: 3,
(c) the polynucleotide sequence of SEQ ID NO: 5,
(d) the polynucleotide sequence of SEQ ID NO: 7,
(e) nucleotides 3590 to 8215 of SEQ ID NO: 2,
(f) nucleotides 3590 to 8341 of SEQ ID NO: 4,
(g) nucleotides 3609 to 6929 of SEQ ID NO: 6,
(h) nucleotides 3590 to 7036 of SEQ ID NO: 8 or
(i) the nucleotide sequence set out in FIG. 13.

14. An rAAV of claim 13, wherein the genome of the rAAV further comprises a muscle-specific transcriptional control element.

15-17. (canceled)

18. An rAAV particle comprising an rAAV of claim 13.

19. A recombinant host cell comprising the polynucleotide of claim 1.

20. (canceled)

21. (canceled)

22. A protein encoded by the polynucleotide of claim 1, wherein the protein comprises an amino acid sequence of SEQ ID NO: 19, SEQ ID NO: 20, SEQ ID NO: 21 or SEQ ID NO: 22.

23. A composition comprising an rAAV particle of claim 18.

24. A method for treating a laminin-deficient muscular dystrophy comprising administering to a patient in need thereof a polynucleotide of claim 1.

25. (canceled)

26. (canceled)

27. The method claim 24 wherein the laminin-deficient muscular dystrophy is MDC1A.

28. A polynucleotide encoding a protein comprising:
a) a first domain comprising the heparin-binding domain of HBEGF and a second domain comprising the processed native alpha chain of the human dystroglycan gene (DAG1alpha); or
b) a first domain comprising the heparin-binding domain of HBEGF and the EGF-like domain of HBEGF, and a second domain comprising DAG1alpha.

29. The polynucleotide of claim 28, wherein the first domain of the protein is encoded by the nucleotide sequence of SEQ ID NO: 13 or SEQ ID NO: 14 or wherein the second domain of the protein is encoded by the nucleotide sequence of SEQ ID NO: 17.

30. (canceled)

31. The polynucleotide of claim 28, wherein the polypeptide comprises (a) the nucleotide sequence of SEQ ID NO: 13 and SEQ ID NO: 17 or (b) the nucleotide sequence of SEQ ID NO: 14 and SEQ ID NO: 17.

32. (canceled)

33. The polynucleotide of claim 28 wherein the polynucleotide of a) comprising i) nucleotides 14 to 1360 set forth in FIG. 7, ii) the nucleotide sequence of SEQ ID NO: 9 or iii) a nucleotide sequence encoding the amino acid sequence of SEQ ID NO: 23 or
wherein the polynucleotide of b) comprising i) nucleotides 14-1486 set forth in FIG. 8, ii) the nucleotide sequence of SEQ ID NO: 11 or iii) a nucleotide sequence encoding the amino acid sequence of SEQ ID NO: 24.

34. (canceled)

35. A recombinant adeno-associate virus (rAAV), wherein the genome of the rAAV comprises the polynucleotide of claim 28.

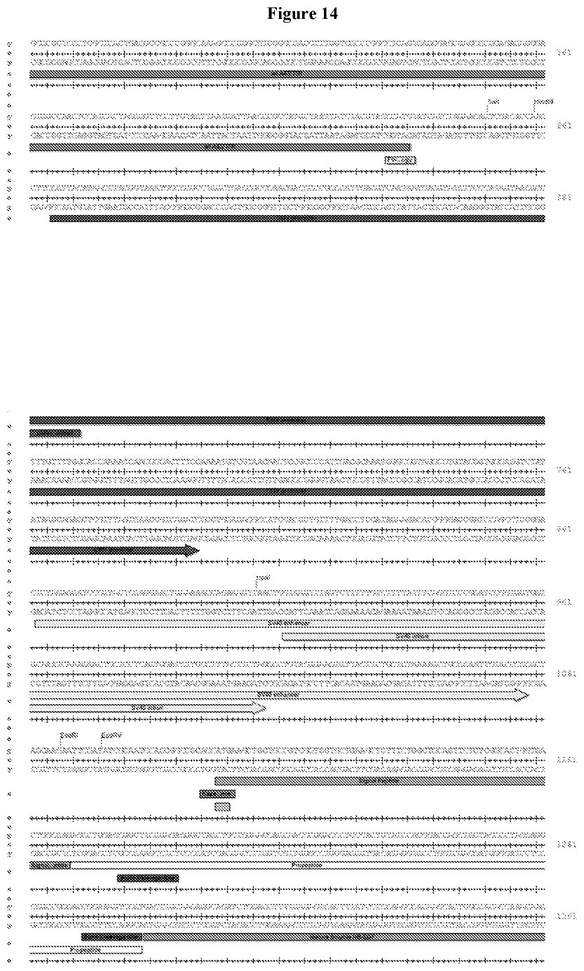

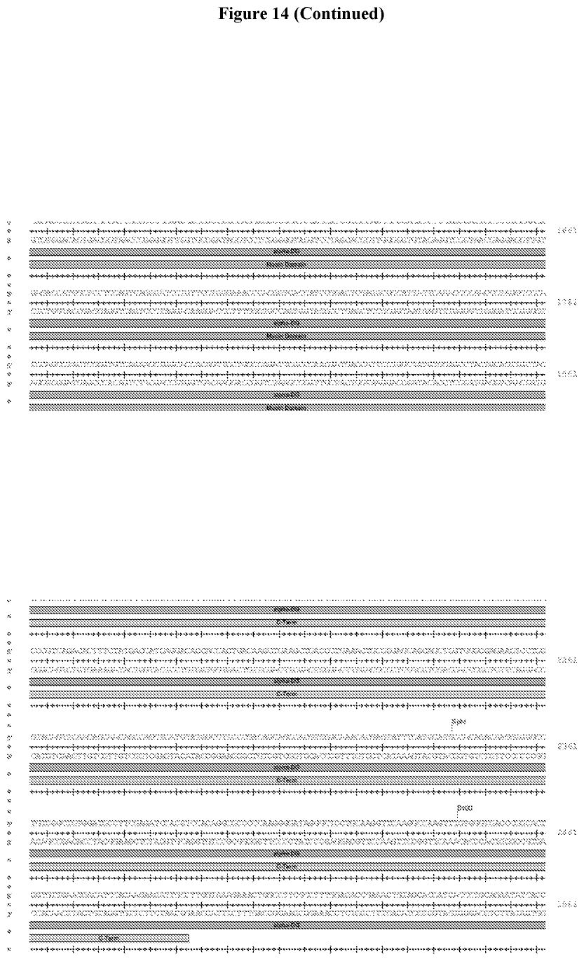

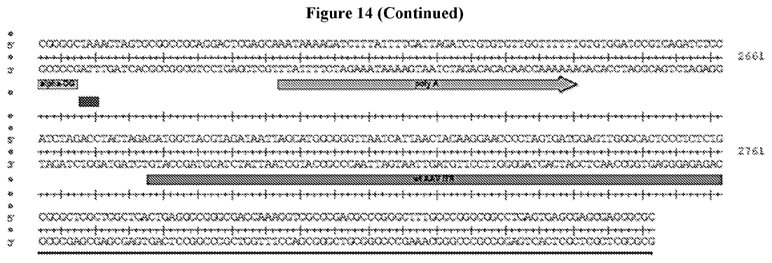

36. A recombinant adeno-associate virus (rAAV), wherein the genome of the rAAV comprises
a) a polynucleotide sequence of SEQ ID NO: 9 or SEQ ID NO: 11,
b) nucleotides 3590 to 6340 of SEQ ID NO: 10,
c) nucleotides 3590 to 6049 of SEQ ID NO: 12 or
d) the nucleotide sequence set out in FIG. 14.

37. An rAAV of claim 36, wherein the genome of the rAAV further comprises a muscle-specific transcriptional control element.

38-43. (canceled)

44. A protein encoded by the polynucleotide of claim 28 wherein the protein comprises the amino acid sequence of SEQ ID NO: 23 or SEQ ID NO: 25.

45. (canceled)

46. A composition comprising a polynucleotide of claim 28.

47. A method for treating a dystroglycanopathy comprising administering to a patient in need thereof a polynucleotide of claim 28.

48. (canceled)

49. (canceled)

50. The method of claim 47 wherein the dystroglycanopathy is Walker Warburg syndrome, Muscle Eye Brain disease, Fukuyama Congenital Muscular Dystrophy, MDC1C, MDC1D, LGMD2I, LGMD2K, LGMD2M, LGMD2N, LGMD2O, LGMD2P, LGMD2T or LGMD2U.


SEQUENCE LISTING (24 sequences)

SEQ ID NO: 1 - DNA, 3222 bp, Artificial Sequence
  Synthetic Polynucleotide
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccaaaagtatctgtgtcttcaggaggtgactgcattcgaacatacaaaccagaaatcaagaaaggaagttacaataatattgttgtcaacgtaaagacagctgttgctgataacctcctcttttatcttggaagtgccaaatttattgactttctggctatagaaatgcgtaaaggcaaagtcagcttcctctgggatgttggatctggagttggacgtgtagagtacccagatttgactattgatgactcatattggtaccgtatcgtagcatcaagaactgggagaaatggaactatttctgtgagagccctggatggacccaaagccagcattgtgcccagcacacaccattcgacgtctcctccagggtacacgattctagatgtggatgcaaatgcaatgctgtttgttggtggcctgactgggaaattaaagaaggctgatgctgtacgtgtgattacattcactggctgcatgggagaaacatactttgacaacaaacctataggtttgtggaatttccgagaaaaagaaggtgactgcaaaggatgcactgtcagtcctcaggtggaagatagtgaggggactattcaatttgatggagaaggttatgcattggtcagccgtcccattcgctggtaccccaacatctccactgtcatgttcaagttcagaacattttcttcgagtgctcttctgatgtatcttgccacacgagacctgagagatttcatgagtgtggagctcactgatgggcacataaaagtcagttacgatctgggctcaggaatggcttccgttgtcagcaatcaaaaccataatgatgggaaatggaaatcattcactctgtcaagaattcaaaaacaagccaatatatcaattgtagatatagatactaatcaggaggagaatatagcaacttcgtcttctggaaacaactttggtcttgacttgaaagcagatgacaaaatatattttggtggcctgccaacgctgagaaacttgagtatgaaagcaaggccagaagtaaatctgaagaaatattccggctgcctcaaagatattgaaatttcaagaactccgtacaatatactcagtagtcccgattatgttggtgttaccaaaggatgttccctggagaatgtttacacagttagctttcctaagcctggttttgtggagctctcccctgtgccaattgatgtaggaacagaaatcaacctgtcattcagcaccaagaatgagtccggcatcattcttttgggaagtggagggacaccagcaccacctaggagaaaacgaaggcagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactga

SEQ ID NO: 2 - DNA, 8270 bp, Artificial Sequence
  Synthetic polynucleotide
  misc_feature: rAAV
  misc_feature (3590)..(3595): n is a, c, g, t or u
  misc_feature (8210)..(8215): n is a, c, g, t or u
gctcttccgcttggtcgctcactgactcgctgcgctcggtcgttcggctgcggcgagcggtatcagctcactcaaacccggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtacgcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaacttccattcgccattcaggctgcgcaactgttgggaagggcgatcggtgcgggcctcttcgctattacgccagctgnnnnnngcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccaaaagtatctgtgtcttcaggaggtgactgcattcgaacatacaaaccagaaatcaagaaaggaagttacaataatattgttgtcaacgtaaagacagctgttgctgataacctcctcttttatcttggaagtgccaaatttattgactttctggctatagaaatgcgtaaaggcaaagtcagcttcctctgggatgttggatctggagttggacgtgtagagtacccagatttgactattgatgactcatattggtaccgtatcgtagcatcaagaactgggagaaatggaactatttctgtgagagccctggatggacccaaagccagcattgtgcccagcacacaccattcgacgtctcctccagggtacacgattctagatgtggatgcaaatgcaatgctgtttgttggtggcctgactgggaaattaaagaaggctgatgctgtacgtgtgattacattcactggctgcatgggagaaacatactttgacaacaaacctataggtttgtggaatttccgagaaaaagaaggtgactgcaaaggatgcactgtcagtcctcaggtggaagatagtgaggggactattcaatttgatggagaaggttatgcattggtcagccgtcccattcgctggtaccccaacatctccactgtcatgttcaagttcagaacattttcttcgagtgctcttctgatgtatcttgccacacgagacctgagagatttcatgagtgtggagctcactgatgggcacataaaagtcagttacgatctgggctcaggaatggcttccgttgtcagcaatcaaaaccataatgatgggaaatggaaatcattcactctgtcaagaattcaaaaacaagccaatatatcaattgtagatatagatactaatcaggaggagaatatagcaacttcgtcttctggaaacaactttggtcttgacttgaaagcagatgacaaaatatattttggtggcctgccaacgctgagaaacttgagtatgaaagcaaggccagaagtaaatctgaagaaatattccggctgcctcaaagatattgaaatttcaagaactccgtacaatatactcagtagtcccgattatgttggtgttaccaaaggatgttccctggagaatgtttacacagttagctttcctaagcctggttttgtggagctctcccctgtgccaattgatgtaggaacagaaatcaacctgtcattcagcaccaagaatgagtccggcatcattcttttgggaagtggagggacaccagcaccacctaggagaaaacgaaggcagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactgaactagtgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaccacatggctacgtagataattagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcnnnnnncagctgcattaatgaatcggccaacgcgcggggagaggcggtttgcgtattgggc

SEQ ID NO: 3 - DNA, 3348 bp, Artificial Sequence
  Synthetic Polynucleotide
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccaaaagtatctgtgtcttcaggaggtgactgcattcgaacatacaaaccagaaatcaagaaaggaagttacaataatattgttgtcaacgtaaagacagctgttgctgataacctcctcttttatcttggaagtgccaaatttattgactttctggctatagaaatgcgtaaaggcaaagtcagcttcctctgggatgttggatctggagttggacgtgtagagtacccagatttgactattgatgactcatattggtaccgtatcgtagcatcaagaactgggagaaatggaactatttctgtgagagccctggatggacccaaagccagcattgtgcccagcacacaccattcgacgtctcctccagggtacacgattctagatgtggatgcaaatgcaatgctgtttgttggtggcctgactgggaaattaaagaaggctgatgctgtacgtgtgattacattcactggctgcatgggagaaacatactttgacaacaaacctataggtttgtggaatttccgagaaaaagaaggtgactgcaaaggatgcactgtcagtcctcaggtggaagatagtgaggggactattcaatttgatggagaaggttatgcattggtcagccgtcccattcgctggtaccccaacatctccactgtcatgttcaagttcagaacattttcttcgagtgctcttctgatgtatcttgccacacgagacctgagagatttcatgagtgtggagctcactgatgggcacataaaagtcagttacgatctgggctcaggaatggcttccgttgtcagcaatcaaaaccataatgatgggaaatggaaatcattcactctgtcaagaattcaaaaacaagccaatatatcaattgtagatatagatactaatcaggaggagaatatagcaacttcgtcttctggaaacaactttggtcttgacttgaaagcagatgacaaaatatattttggtggcctgccaacgctgagaaacttgagtatgaaagcaaggccagaagtaaatctgaagaaatattccggctgcctcaaagatattgaaatttcaagaactccgtacaatatactcagtagtcccgattatgttggtgttaccaaaggatgttccctggagaatgtttacacagttagctttcctaagcctggttttgtggagctctcccctgtgccaattgatgtaggaacagaaatcaacctgtcattcagcaccaagaatgagtccggcatcattcttttgggaagtggagggacaccagcaccacctaggagaaaacgaaggcagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactga

SEQ ID NO: 4 - DNA, 8396 bp, Artificial Sequence
  Synthetic Polynucleotide
  misc_feature: rAAV
  misc_feature (3590)..(3595): n is a, c, g, t or u
  misc_feature (8336)..(8341): n is a, c, g, t or u
gctcttccgcttggtcgctcactgactcgctgcgctcggtcgttcggctgcggcgagcggtatcagctcactcaaacccggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtacgcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaacttccattcgccattcaggctgcgcaactgttgggaagggcgatcggtgcgggcctcttcgctattacgccagctgnnnnnngcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccaaaagtatctgtgtcttcaggaggtgactgcattcgaacatacaaaccagaaatcaagaaaggaagttacaataatattgttgtcaacgtaaagacagctgttgctgataacctcctcttttatcttggaagtgccaaatttattgactttctggctatagaaatgcgtaaaggcaaagtcagcttcctctgggatgttggatctggagttggacgtgtagagtacccagatttgactattgatgactcatattggtaccgtatcgtagcatcaagaactgggagaaatggaactatttctgtgagagccctggatggacccaaagccagcattgtgcccagcacacaccattcgacgtctcctccagggtacacgattctagatgtggatgcaaatgcaatgctgtttgttggtggcctgactgggaaattaaagaaggctgatgctgtacgtgtgattacattcactggctgcatgggagaaacatactttgacaacaaacctataggtttgtggaatttccgagaaaaagaaggtgactgcaaaggatgcactgtcagtcctcaggtggaagatagtgaggggactattcaatttgatggagaaggttatgcattggtcagccgtcccattcgctggtaccccaacatctccactgtcatgttcaagttcagaacattttcttcgagtgctcttctgatgtatcttgccacacgagacctgagagatttcatgagtgtggagctcactgatgggcacataaaagtcagttacgatctgggctcaggaatggcttccgttgtcagcaatcaaaaccataatgatgggaaatggaaatcattcactctgtcaagaattcaaaaacaagccaatatatcaattgtagatatagatactaatcaggaggagaatatagcaacttcgtcttctggaaacaactttggtcttgacttgaaagcagatgacaaaatatattttggtggcctgccaacgctgagaaacttgagtatgaaagcaaggccagaagtaaatctgaagaaatattccggctgcctcaaagatattgaaatttcaagaactccgtacaatatactcagtagtcccgattatgttggtgttaccaaaggatgttccctggagaatgtttacacagttagctttcctaagcctggttttgtggagctctcccctgtgccaattgatgtaggaacagaaatcaacctgtcattcagcaccaagaatgagtccggcatcattcttttgggaagtggagggacaccagcaccacctaggagaaaacgaaggcagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactgaactagtgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaccacatggctacgtagataattagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcnnnnnncagctgcattaatgaatcggccaacgcgcggggagaggcggtttgcgtattgggc

SEQ ID NO: 5 - DNA, 1917 bp, Artificial Sequence
  Synthetic Polynucleotide
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccacagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactga

SEQ ID NO: 6 - DNA, 6984 bp, Artificial Sequence
  Synthetic Polynucleotide
  misc_feature: rAAV
  misc_feature (3609)..(3614): n is a, c, g, t or u
  misc_feature (6924)..(6929): n is a, c, g, t or u
gctcttccgcttggtcgctcactgactcgctgcgctcggtcgttcggctgcggcgagcggtatcagctcactcaaacccggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtacgcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccatcgttacataacttacggtatattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaacttccattcgccattcaggctgcgcaactgttgggaagggcgatcggtgcgggcctcttcgctattacgccagctgnnnnnngcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccacagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactgaactagtgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaccacatggctacgtagataattagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcnnnnnncagctgcattaatgaatcggccaacgcgcggggagaggcggtttgcgtattgggc

SEQ ID NO: 7 - DNA, 2043 bp, Artificial Sequence
  Synthetic Polynucleotide
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccacagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactga

SEQ ID NO: 8 - DNA, 7091 bp, Artificial Sequence
  Synthetic Polynucleotide
  misc_feature: rAAV
  misc_feature (3590)..(3595): n is a, c, g, t or u
  misc_feature (7031)..(7036): n is a, c, g, t or u
gctcttccgcttggtcgctcactgactcgctgcgctcggtcgttcggctgcggcgagcggtatcagctcactcaaacccggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtacgcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaacttccattcgccattcaggctgcgcaactgttgggaagggcgatcggtgcgggcctcttcgctattacgccagctgnnnnnngcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccacagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactgaactagtgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaccacatggctacgtagataattagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcnnnnnncagctgcattaatgaatcggccaacgcgcggggagaggcggtttgcgtattgggc

SEQ ID NO: 9 - DNA, 1347 bp, Artificial Sequence
  Synthetic Polynucleotide
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccacagatccatgctacacccacacctgtcactgccattgggcccccaaccacggctatccaggagcccccatccaggatcgtgccaacccccacatctccagccattgctcctccaacagagaccatggctcctccagtcagggatcctgttcctgggaaacccacggtcaccatccggactcgaggcgccattattcaaaccccaaccctaggccccatccagcctactcgggtgtcagaagctggcaccacagttcctggccagattcgcccaacgatgaccattcctggctatgtggagcctactgcagttgctacccctcccacaaccaccaccaagaagccacgagtatccacaccaaaaccagcaacgccttcaactgactccaccaccaccacgactcgcaggccaaccaagaaaccacggacaccccggccagtgccccgggtcaccaccaaagtttccatcaccagattggaaactgcctcaccgcctactcgtattcgcaccaccaccagtggagtgccccgtggcggagaacccaaccagcgcccagagctcaagaaccatattgacagggtagatgcctgggttggcacctactttgaggtgaagatcccgtcagacactttctatgaccatgaggacaccaccactgacaagctgaagctgaccctgaaactgcgggagcagcagctggtgggcgagaagtcctgggtacagttcaacagcaacagccagctcatgtatggccttcccgacagcagccacgtgggcaaacacgagtatttcatgcatgccacagacaaggggggcctgtcggctgtggatgccttcgagatccacgtccacaggcgcccccaaggggatagggctcctgcaaggttcaaggccaagtttgtgggtgacccggcactggtgttgaatgacatccacaagaagattgccttggtaaagaaactggccttcgcctttggagaccgaaactgtagcaccatcaccctgcagaatatcacccggggctaa

SEQ ID NO: 10 - DNA, 6395 bp, Artificial Sequence
  Synthetic Polynucleotide
  misc_feature: rAAV
  misc_feature (3590)..(3595): n is a, c, g, t or u
  misc_feature (6335)..(6340): n is a, c, g, t or u
gctcttccgcttggtcgctcactgactcgctgcgctcggtcgttcggctgcggcgagcggtatcagctcactcaaacccggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtacgcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaacttccattcgccattcaggctgcgcaactgttgggaagggcgatcggtgcgggcctcttcgctattacgccagctgnnnnnngcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccacagatccatgctacacccacacctgtcactgccattgggcccccaaccacggctatccaggagcccccatccaggatcgtgccaacccccacatctccagccattgctcctccaacagagaccatggctcctccagtcagggatcctgttcctgggaaacccacggtcaccatccggactcgaggcgccattattcaaaccccaaccctaggccccatccagcctactcgggtgtcagaagctggcaccacagttcctggccagattcgcccaacgatgaccattcctggctatgtggagcctactgcagttgctacccctcccacaaccaccaccaagaagccacgagtatccacaccaaaaccagcaacgccttcaactgactccaccaccaccacgactcgcaggccaaccaagaaaccacggacaccccggccagtgccccgggtcaccaccaaagtttccatcaccagattggaaactgcctcaccgcctactcgtattcgcaccaccaccagtggagtgccccgtggcggagaacccaaccagcgcccagagctcaagaaccatattgacagggtagatgcctgggttggcacctactttgaggtgaagatcccgtcagacactttctatgaccatgaggacaccaccactgacaagctgaagctgaccctgaaactgcgggagcagcagctggtgggcgagaagtcctgggtacagttcaacagcaacagccagctcatgtatggccttcccgacagcagccacgtgggcaaacacgagtatttcatgcatgccacagacaaggggggcctgtcggctgtggatgccttcgagatccacgtccacaggcgcccccaaggggatagggctcctgcaaggttcaaggccaagtttgtgggtgacccggcactggtgttgaatgacatccacaagaagattgccttggtaaagaaactggccttcgcctttggagaccgaaactgtagcaccatcaccctgcagaatatcacccggggctaaactagtgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaccacatggctacgtagataattagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcnnnnnncagctgcattaatgaatcggccaacgcgcggggagaggcggtttgcgtattgggc

SEQ ID NO: 11 - DNA, 1473 bp, Artificial Sequence
  Synthetic Polynucleotide
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccacagatccatgctacacccacacctgtcactgccattgggcccccaaccacggctatccaggagcccccatccaggatcgtgccaacccccacatctccagccattgctcctccaacagagaccatggctcctccagtcagggatcctgttcctgggaaacccacggtcaccatccggactcgaggcgccattattcaaaccccaaccctaggccccatccagcctactcgggtgtcagaagctggcaccacagttcctggccagattcgcccaacgatgaccattcctggctatgtggagcctactgcagttgctacccctcccacaaccaccaccaagaagccacgagtatccacaccaaaaccagcaacgccttcaactgactccaccaccaccacgactcgcaggccaaccaagaaaccacggacaccccggccagtgccccgggtcaccaccaaagtttccatcaccagattggaaactgcctcaccgcctactcgtattcgcaccaccaccagtggagtgccccgtggcggagaacccaaccagcgcccagagctcaagaaccatattgacagggtagatgcctgggttggcacctactttgaggtgaagatcccgtcagacactttctatgaccatgaggacaccaccactgacaagctgaagctgaccctgaaactgcgggagcagcagctggtgggcgagaagtcctgggtacagttcaacagcaacagccagctcatgtatggccttcccgacagcagccacgtgggcaaacacgagtatttcatgcatgccacagacaaggggggcctgtcggctgtggatgccttcgagatccacgtccacaggcgcccccaaggggatagggctcctgcaaggttcaaggccaagtttgtgggtgacccggcactggtgttgaatgacatccacaagaagattgccttggtaaagaaactggccttcgcctttggagaccgaaactgtagcaccatcaccctgcagaatatcacccggggctaa

SEQ ID NO: 12 - DNA, 6521 bp, Artificial Sequence
  Synthetic Polynucleotide
  misc_feature: rAAV
  misc_feature (3590)..(3595): n is a, c, g, t or u
  misc_feature (6461)..(6466): n is a, c, g, t or u
gctcttccgcttggtcgctcactgactcgctgcgctcggtcgttcggctgcggcgagcggtatcagctcactcaaacccggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtacgcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaacttccattcgccattcaggctgcgcaactgttgggaagggcgatcggtgcgggcctcttcgctattacgccagctgnnnnnngcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccacagatccatgctacacccacacctgtcactgccattgggcccccaaccacggctatccaggagcccccatccaggatcgtgccaacccccacatctccagccattgctcctccaacagagaccatggctcctccagtcagggatcctgttcctgggaaacccacggtcaccatccggactcgaggcgccattattcaaaccccaaccctaggccccatccagcctactcgggtgtcagaagctggcaccacagttcctggccagattcgcccaacgatgaccattcctggctatgtggagcctactgcagttgctacccctcccacaaccaccaccaagaagccacgagtatccacaccaaaaccagcaacgccttcaactgactccaccaccaccacgactcgcaggccaaccaagaaaccacggacaccccggccagtgccccgggtcaccaccaaagtttccatcaccagattggaaactgcctcaccgcctactcgtattcgcaccaccaccagtggagtgccccgtggcggagaacccaaccagcgcccagagctcaagaaccatattgacagggtagatgcctgggttggcacctactttgaggtgaagatcccgtcagacactttctatgaccatgaggacaccaccactgacaagctgaagctgaccctgaaactgcgggagcagcagctggtgggcgagaagtcctgggtacagttcaacagcaacagccagctcatgtatggccttcccgacagcagccacgtgggcaaacacgagtatttcatgcatgccacagacaaggggggcctgtcggctgtggatgccttcgagatccacgtccacaggcgcccccaaggggatagggctcctgcaaggttcaaggccaagtttgtgggtgacccggcactggtgttgaatgacatccacaagaagattgccttggtaaagaaactggccttcgcctttggagaccgaaactgtagcaccatcaccctgcagaatatcacccggggctaaactagtgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaccacatggctacgtagataattagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcnnnnnncagctgcattaatgaatcggccaacgcgcggggagaggcggtttgcgtattgggc

SEQ ID NO: 13 - DNA, 321 bp, Artificial Sequence
  Synthetic Polynucleotide
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggaccca

SEQ ID NO: 14 - DNA, 447 bp, Artificial Sequence
  Synthetic Polynucleotide
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctccca

SEQ ID NO: 15 - DNA, 2901 bp, Artificial Sequence
  Synthetic Polynucleotide
aaagtatctgtgtcttcaggaggtgactgcattcgaacatacaaaccagaaatcaagaaaggaagttacaataatattgttgtcaacgtaaagacagctgttgctgataacctcctcttttatcttggaagtgccaaatttattgactttctggctatagaaatgcgtaaaggcaaagtcagcttcctctgggatgttggatctggagttggacgtgtagagtacccagatttgactattgatgactcatattggtaccgtatcgtagcatcaagaactgggagaaatggaactatttctgtgagagccctggatggacccaaagccagcattgtgcccagcacacaccattcgacgtctcctccagggtacacgattctagatgtggatgcaaatgcaatgctgtttgttggtggcctgactgggaaattaaagaaggctgatgctgtacgtgtgattacattcactggctgcatgggagaaacatactttgacaacaaacctataggtttgtggaatttccgagaaaaagaaggtgactgcaaaggatgcactgtcagtcctcaggtggaagatagtgaggggactattcaatttgatggagaaggttatgcattggtcagccgtcccattcgctggtaccccaacatctccactgtcatgttcaagttcagaacattttcttcgagtgctcttctgatgtatcttgccacacgagacctgagagatttcatgagtgtggagctcactgatgggcacataaaagtcagttacgatctgggctcaggaatggcttccgttgtcagcaatcaaaaccataatgatgggaaatggaaatcattcactctgtcaagaattcaaaaacaagccaatatatcaattgtagatatagatactaatcaggaggagaatatagcaacttcgtcttctggaaacaactttggtcttgacttgaaagcagatgacaaaatatattttggtggcctgccaacgctgagaaacttgagtatgaaagcaaggccagaagtaaatctgaagaaatattccggctgcctcaaagatattgaaatttcaagaactccgtacaatatactcagtagtcccgattatgttggtgttaccaaaggatgttccctggagaatgtttacacagttagctttcctaagcctggttttgtggagctctcccctgtgccaattgatgtaggaacagaaatcaacctgtcattcagcaccaagaatgagtccggcatcattcttttgggaagtggagggacaccagcaccacctaggagaaaacgaaggcagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactga

SEQ ID NO: 16 - DNA, 1332 bp, Artificial Sequence
  Synthetic Polynucleotide
cagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatgga

SEQ ID NO: 17 - DNA, 1023 bp, Artificial Sequence
  Synthetic Polynucleotide
cagatccatgctacacccacacctgtcactgccattgggcccccaaccacggctatccaggagcccccatccaggatcgtgccaacccccacatctccagccattgctcctccaacagagaccatggctcctccagtcagggatcctgttcctgggaaacccacggtcaccatccggactcgaggcgccattattcaaaccccaaccctaggccccatccagcctactcgggtgtcagaagctggcaccacagttcctggccagattcgcccaacgatgaccattcctggctatgtggagcctactgcagttgctacccctcccacaaccaccaccaagaagccacgagtatccacaccaaaaccagcaacgccttcaactgactccaccaccaccacgactcgcaggccaaccaagaaaccacggacaccccggccagtgccccgggtcaccaccaaagtttccatcaccagattggaaactgcctcaccgcctactcgtattcgcaccaccaccagtggagtgccccgtggcggagaacccaaccagcgcccagagctcaagaaccatattgacagggtagatgcctgggttggcacctactttgaggtgaagatcccgtcagacactttctatgaccatgaggacaccaccactgacaagctgaagctgaccctgaaactgcgggagcagcagctggtgggcgagaagtcctgggtacagttcaacagcaacagccagctcatgtatggccttcccgacagcagccacgtgggcaaacacgagtatttcatgcatgccacagacaaggggggcctgtcggctgtggatgccttcgagatccacgtccacaggcgcccccaaggggatagggctcctgcaaggttcaaggccaagtttgtgggtgacccggcactggtgttgaatgacatccacaagaagattgccttggtaaagaaactggccttcgcctttggagaccgaaactgtagcaccatcaccctgcagaatatcacccggggc

SEQ ID NO: 18 - DNA, 529 bp, Artificial Sequence
  Synthetic Polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatc

SEQ ID NO: 19 - protein, 1073 aa, Artificial Sequence
  Synthetic Polypeptide
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPKVSVSSGGDCIRTYKPEIKKGSYNNIVVNVKTAVADNLLFYLGSAKFIDFLAIEMRKGKVSFLWDVGSGVGRVEYPDLTIDDSYWYRIVASRTGRNGTISVRALDGPKASIVPSTHHSTSPPGYTILDVDANAMLFVGGLTGKLKKADAVRVITFTGCMGETYFDNKPIGLWNFREKEGDCKGCTVSPQVEDSEGTIQFDGEGYALVSRPIRWYPNISTVMFKFRTFSSSALLMYLATRDLRDFMSVELTDGHIKVSYDLGSGMASVVSNQNHNDGKWKSFTLSRIQKQANISIVDIDTNQEENIATSSSGNNFGLDLKADDKIYFGGLPTLRNLSMKARPEVNLKKYSGCLKDIEISRTPYNILSSPDYVGVTKGCSLENVYTVSFPKPGFVELSPVPIDVGTEINLSFSTKNESGIILLGSGGTPAPPRRKRRQTGQAYYVILLNRGRLEVHLSTGARTMRKIVIRPEPNLFHDGREHSVHVERTRGIFTVQVDENRRYMQNLTVEQPIEVKKLFVGGAPPEFQPSPLRNIPPFEGCIWNLVINSVPMDFARPVSFKNADIGRCAHQKLREDEDGAAPAEIVIQPEPVPTPAFPTPTPVLTHGPCAAESEPALLIGSKQFGLSRNSHIAIAFDDTKVKNRLTIELEVRTEAESGLLFYMARINHADFATVQLRNGLPYFSYDLGSGDTHTMIPTKINDGQWHKIKIMRSKQEGILYVDGASNRTISPKKADILDVVGMLYVGGLPINYTTRRIGPVTYSIDGCVRNLHMAEAPADLEQPTSSFHVGTCFANAQRGTYFDGTGFAKAVGGFKVGLDLLVEFEFRTTTTTGVLLGISSQKMDGMGIEMIDEKLMFHVDNGAGRFTAVYDAGVPGHLCDGQWHKVTANKIKHRIELTVDGNQVEAQSPNPASTSADTNDPVFVGGFPDDLKQFGLTTSIPFRGCIRSLKLTKGTASHWRLILPRPWN

SEQ ID NO: 20 - protein, 1115 aa, Artificial Sequence
  Synthetic Polypeptide
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPKVSVSSGGDCIRTYKPEIKKGSYNNIVVNVKTAVADNLLFYLGSAKFIDFLAIEMRKGKVSFLWDVGSGVGRVEYPDLTIDDSYWYRIVASRTGRNGTISVRALDGPKASIVPSTHHSTSPPGYTILDVDANAMLFVGGLTGKLKKADAVRVITFTGCMGETYFDNKPIGLWNFREKEGDCKGCTVSPQVEDSEGTIQFDGEGYALVSRPIRWYPNISTVMFKFRTFSSSALLMYLATRDLRDFMSVELTDGHIKVSYDLGSGMASVVSNQNHNDGKWKSFTLSRIQKQANISIVDIDTNQEENIATSSSGNNFGLDLKADDKIYFGGLPTLRNLSMKARPEVNLKKYSGCLKDIEISRTPYNILSSPDYVGVTKGCSLENVYTVSFPKPGFVELSPVPIDVGTEINLSFSTKNESGIILLGSGGTPAPPRRKRRQTGQAYYVILLNRGRLEVHLSTGARTMRKIVIRPEPNLFHDGREHSVHVERTRGIFTVQVDENRRYMQNLTVEQPIEVKKLFVGGAPPEFQPSPLRNIPPFEGCIWNLVINSVPMDFARPVSFKNADIGRCAHQKLREDEDGAAPAEIVIQPEPVPTPAFPTPTPVLTHGPCAAESEPALLIGSKQFGLSRNSHIAIAFDDTKVKNRLTIELEVRTEAESGLLFYMARINHADFATVQLRNGLPYFSYDLGSGDTHTMIPTKINDGQWHKIKIMRSKQEGILYVDGASNRTISPKKADILDVVGMLYVGGLPINYTTRRIGPVTYSIDGCVRNLHMAEAPADLEQPTSSFHVGTCFANAQRGTYFDGTGFAKAVGGFKVGLDLLVEFEFRTTTTTGVLLGISSQKMDGMGIEMIDEKLMFHVDNGAGRFTAVYDAGVPGHLCDGQWHKVTANKIKHRIELTVDGNQVEAQSPNPASTSADTNDPVFVGGFPDDLKQFGLTTSIPFRGCIRSLKLTKGTASHWRLILPRPWN

SEQ ID NO: 21 - protein, 680 aa, Artificial Sequence
  Synthetic Polypeptide
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPQTGQAYYVILLNRGRLEVHLSTGARTMRKIVIRPEPNLFHDGREHSVHVERTRGIFTVQVDENRRYMQNLTVEQPIEVKKLFVGGAPPEFQPSPLRNIPPFEGCIWNLVINSVPMDFARPVSFKNADIGRCAHQKLREDEDGAAPAEIVIQPEPVPTPAFPTPTPVLTHGPCAAESEPALLIGSKQFGLSRNSHIAIAFDDTKVKNRLTIELEVRTEAESGLLFYMARINHADFATVQLRNGLPYFSYDLGSGDTHTMIPTKINDGQWHKIKIMRSKQEGILYVDGASNRTISPKKADILDVVGMLYVGGLPINYTTRRIGPVTYSIDGCVRNLHMAEAPADLEQPTSSFHVGTCFANAQRGTYFDGTGFAKAVGGFKVGLDLLVEFEFRTTTTTGVLLGISSQKMDGMGIEMIDEKLMFHVDNGAGRFTAVYDAGVPGHLCDGQWHKVTANKIKHRIELTVDGNQVEAQSPNPASTSADTNDPVFVGGFPDDLKQFGLTTSIPFRGCIRSLKLTKGTASHWRLILPRPWN

SEQ ID NO: 22 - protein, 680 aa, Artificial Sequence; identical to SEQ ID NO: 21
  Synthetic Polypeptide

SEQ ID NO: 23 - protein, 448 aa, Artificial Sequence
  Synthetic Polypeptide
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRG

SEQ ID NO: 24 - protein, 490 aa, Artificial Sequence
  Synthetic Polypeptide
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRG